(19) United States
(12) Patent Application Publication
(10) Pub. No.: US 2026/0360548 A1
(43) Pub. Date: Nov. 12, 2026

(54) SYSTEMS AND METHODS FOR PRIORITIZING MEDICAL RESOURCES FOR CANCER SCREENING

(71) Applicant: Johnson & Johnson Enterprise Innovation Inc., New Brunswick, NY (US)

(72) Inventors: Michael Schroeder, New York, NY (US); Matthew Mailman, New Hope, PA (US); Anderson Nnewihe, Walpole, MA (US); Takahiro Sato, Philadelphia, PA (US); Robert Malone, Manitou Springs, CO (US); Kirk Solo, Colorado Springs, CO (US)

(73) Assignee: Johnson & Johnson Enterprise Innovation Inc., New Brunswick, NY (US)

(21) Appl. No.: 19/129,017

(22) PCT Filed: Nov. 13, 2023
(86) PCT No.: PCT/IB2023/061440

§ 371 Date: May 11, 2025

Related U.S. Application Data
(60) Provisional application No. 63/424,570, filed on Nov. 11, 2022.

(51) Int. Cl. G16H 50/30; A61B 5/00; G16H 10/60

(52) U.S. Cl. CPC G16H 50/30; A61B 5/7275; A61B 5/742; G16H 10/60

(57) ABSTRACT

Methods involve deploying trained risk prediction models to analyze electronic records of patients to identify which patients are at risk of developing cancer. Electronic records are obtainable in large quantities and in a cost-effective manner and therefore, can be valuable data for continuous monitoring and evaluation of large patient populations for patients that are at risk of developing cancer. Patients at risk of developing cancer, referred to herein as candidate patients, can be prioritized for medical interventions, thereby enabling healthcare providers to appropriate divert medical attention to candidate patients that are most in need. Candidate patients can undergo subsequent imaging and/or biopsy procedures to confirm the risk of developing cancer.

### CROSS-REFERENCE TO RELATED APPLICATION

This application claims priority to and the benefit of U.S. Provisional Application No. 63/424,570, filed Nov. 11, 2022, of which is incorporated by reference herein in its entirety.

### BACKGROUND

Lung cancer most commonly begins with the development of a lung nodule. Generally, the larger the nodule, the more rapid its growth or the more irregular it is in appearance, and the more likely it is to be cancer. In many scenarios, lung nodules in patients remain undetected for periods of time or, even if detected, can already indicate an advanced stage of cancer. Thus, early prediction of lung cancer risk in patients even prior to the development of one or more lung nodules can be valuable. However, early-stage cancer screening remains difficult as screening large numbers of patients using resource-intensive methodologies would be infeasible. For example, performing tissue biopsies and/or image scanning across large patient populations is untenable. Therefore, there is a need to effectively identify patients most likely at risk of developing cancer.

### SUMMARY

Embodiments of the disclosure disclosed herein involve implementing machine learning models to analyze electronic records of patients. Electronic records of patients can represent valuable information that are predictive for the risk of cancer. Furthermore, electronic records can be accumulated easily and cost effectively e.g., during patient visits. Therefore, electronic records can be valuable data for continuous monitoring and evaluation of patients for their risk of developing cancer. Methods disclosed herein are useful for identifying such patients that may be at risk of developing cancer, hereafter referred to as candidate patients. Therefore, healthcare providers, who may be caring for large numbers of patients, can appropriately prioritize limited medical resources by providing interventions to candidate patients that are at most risk of developing cancer. For example, candidate patients can undergo subsequent imaging and/or biopsy procedures, which are far more costly procedures for confirming whether the candidate patients are at risk of developing cancer.

Disclosed herein is a method for prioritizing medical resources for screening a patient for cancer, the method comprising obtaining a temporally diverse dataset comprising electronic records of a patient; weighting features from data of the electronic records of the patient according to timepoints that the data were recorded in the electronic records of the patient; analyzing the weighted features for the patient using a machine learning model to categorize the patient as a candidate patient at risk for cancer or a non-candidate patient; and responsive to determining the patient as a candidate patient, providing identification of the candidate patient for prioritization of medical resources.

In various embodiments, weighting the features according to timepoints that the data were recorded in the electronic records of the patient comprises assigning higher weights to features from data that were more recently recorded in the electronic records in comparison to features from data that were earlier recorded in the electronic records. In various embodiments, methods disclosed herein further comprise normalizing the data of the electronic records of the patient. In various embodiments, normalizing the data comprises applying a hyperbolic tangent transformation. In various embodiments, the machine learning model outputs a score indicative of cancer risk for the patient. In various embodiments, the score indicative of cancer risk is a continuous score between 0 and 1. In various embodiments, the machine learning model further outputs an identification of a feature or feature grouping that contributed to the score indicative of cancer risk.

In various embodiments, providing identification of the candidate patient for prioritization of medical resources further comprises providing the corresponding identifications of features or feature groupings of the candidate patient for prioritization of medical resources. In various embodiments, the features from data of the electronic records comprises features from electronic health record (EHR) data. In various embodiments, the features from data of the electronic records comprises features from medical claims data. In various embodiments, the features from data of the electronic records comprises features from EHR data and medical claims data. In various embodiments, the features from EHR data comprises one or more of patient demographics data, laboratory test data, prior diagnoses data, prior procedures data, and prior prescriptions data. In various embodiments, the patient demographics data comprises one or more of patient gender, patient age, patient behavior, patient ethnicity, patient race, patient geographic location, patient socioeconomic status, education status, number of months active, and/or insurance status. In various embodiments, the patient behavior comprises smoking behavior selected from one of current smoker, previously smoked, never smoked, other smoking, not currently smoking, or unknown smoke. In various embodiments, the features from medical claims data comprises one or more of patient demographics data, prior diagnoses data, prior procedures data, and prior prescriptions data.

In various embodiments, the prior diagnoses data comprises one or more diagnostic codes. In various embodiments, the one or more diagnostic codes comprise ICD-9 or ICD-10 codes. In various embodiments, the one or more diagnostic codes comprise ICD-10 codes, wherein one or more ICD-10 codes were converted from one or more ICD-9 codes. In various embodiments, the prior procedures data comprises one or more procedures codes. In various embodiments, the one or more procedures codes comprise HCPCS or CPT-4 codes. In various embodiments, the prior prescriptions data comprises one or more national drug codes (NDCs). In various embodiments, the patient is between 50-80 years old. In various embodiments, the patient exhibits a prior smoking history. In various embodiments, the patient has not previously undergone a computed tomography (CT) scan, a positron emission tomography (PET) scan, or a PET-CT scan. In various embodiments, the patient has not previously undergone a cancer biopsy procedure. In various embodiments, the patient has not previously received a cancer diagnosis.

In various embodiments, the cancer comprises lung cancer. In various embodiments, the lung cancer is one of non-small cell lung cancer, small cell lung cancer, adenocarcinoma, and squamous cell carcinoma. In various embodiments, the prioritization of medical resources comprises prioritizing patients for undergoing computed tomography (CT) scans. In various embodiments, the machine learning model comprises a logistic regression model, a random forest model, or a neural network.

In various embodiments, methods disclosed herein further comprise obtaining updated electronic records for one or more patients, the updated electronic records comprising additional data recorded in the updated electronic records subsequent to providing identification of the candidate patient; analyzing features from the additional data using a machine learning model to categorize a patient as an additional candidate patient at risk for cancer or a non-candidate patient; and responsive to determining that the patient is an additional candidate patient, providing identification of the additional candidate patient for prioritization of medical resources.

Additionally disclosed herein is a method for prioritizing medical resources for screening a patient for cancer, the method comprising obtaining a dataset comprising electronic records of a patient; receiving an indication of available medical resources of a third party; extracting features from data of the electronic records of the patient; analyzing the extracted features for the patient using a machine learning model to categorize the patient as a candidate patient at risk for cancer or a non-candidate patient, wherein the categorizing of the patient uses at least a prediction of the machine learning model and a threshold selected according to the received indication; and responsive to determining the patient as a candidate patient, providing identification of the candidate patient for prioritization of medical resources. In various embodiments, the threshold is selected to account for the available medical resources of the third party. In various embodiments, a lower threshold is selected for the third party for an indication reflecting higher available medical resources of the third party, in comparison to a higher threshold that is selected for the third party for an indication reflecting lower available resources for the third party.

In various embodiments, methods disclosed herein further comprise weighting the extracted features according to timepoints that the data were recorded in the electronic records of the patient.

Additionally disclosed herein is a method for prioritizing medical resources for screening individuals for cancer, the method comprising obtaining a dataset comprising electronic records of a patient; extracting features from data of the electronic records of the patient; analyzing the extracted features for the patient using a machine learning model to categorize the patient as a candidate patient at risk for cancer or a non-candidate patient, wherein the machine learning model is configured to output 1) a score indicative of lung cancer risk for the patient and 2) identification of a feature grouping that contributed to the score indicative of lung cancer risk, wherein the feature grouping comprises two or more features; and responsive to determining the patient as a candidate patient, providing identification of the candidate patient and the identification of the feature grouping to the third party for prioritization of medical resources.

In various embodiments, the feature grouping comprises at least 2 features. In some embodiments, the feature grouping may comprise between 2 and 10 features. In various embodiments, the feature grouping comprises one of a lung issue grouping, heart issue grouping, smoking status grouping, patient characteristics grouping, patient behavior grouping, and vaccine grouping. In various embodiments, the lung issue grouping comprises one or more of chronic obstructive pulmonary disease (COPD), chronic bronchitis, pleural effusion, dyspnea, wheezing, and inhaled treatment for COPD and/or asthma. In various embodiments, the heart issue grouping comprises one or more of atherosclerotic heart disease, iron deficiency anemias, elevated blood pressure, treatment for high blood pressure, and treatment for reducing risk of heart attack and/or stroke. In various embodiments, the smoking status grouping comprises one or more of tobacco use, nicotine dependence, cigarette use, smoking cessation, number of months actively smoking, never smoked observation, and current smoker observation. In various embodiments, the patient characteristics grouping comprises one or more of systolic blood pressure, diastolic blood pressure, number of months active, patient age, and geographic location. In various embodiments, the patient behavior grouping comprises one or more of prior established patient visits and new patient visits. In various embodiments, the vaccine grouping comprises one or more of pneumonia vaccine and flu vaccine. In various embodiments, analyzing the extracted features using the machine learning model comprises implementing a Shapley additive contribution algorithm to determine contributions of one or more feature groupings. In various embodiments, the feature grouping identified by the output of the machine learning model comprises a feature grouping providing the highest contribution to the score. In various embodiments, the output of the machine learning model further comprises an identification of a second feature grouping providing the second highest contribution to the score. In various embodiments, the output of the machine learning model further comprises an identification of a third feature grouping providing the third highest contribution to the score. In various embodiments, categorizing the patient as a candidate patient or a non-candidate patient based on the score further comprises selecting a threshold according to a received indication of available medical resources of the third party; and categorizing the patient as a candidate patient or a non-candidate patient using the score and the threshold.

### BRIEF DESCRIPTION OF THE DRAWINGS

These and other features, aspects, and advantages of the present disclosure will become better understood with regard to the following description and accompanying drawings.

FIG. 1A depicts a system overview for prioritizing medical resources for candidate patients, in accordance with an embodiment.

FIG. 1B depicts a block diagram of an example patient prioritization system, in accordance with an embodiment.

FIG. 2A depicts an example flow diagram for implementing a risk prediction model for identifying candidate patients, in accordance with an embodiment.

FIG. 2B depicts an example diagram for organizing the electronic data, in accordance with an embodiment.

FIG. 3A depicts an example flow process for identifying candidate patients, in accordance with a first embodiment.

FIG. 3B depicts an example flow process for identifying candidate patients, in accordance with a second embodiment.

FIG. 3C depicts an example interaction diagram for identifying candidate patients, in accordance with a third embodiment.

FIG. 4 illustrates an example computer for implementing the entities shown in FIGS. 1A, 1B, 2, 3A, and 3B.

FIG. 5A depicts an example data pipeline for developing the algorithm (e.g., machine learning model).

FIG. 5B depicts an example data pipeline for validating the algorithm (e.g., machine learning model).

FIG. 6A depicts example output for a patient with a standard lung cancer risk.

FIG. 6B depicts example output for a patient with an elevated lung cancer risk.

FIG. 7 shows performance of various machine learning models.

### DETAILED DESCRIPTION

### I. DEFINITIONS

Terms used in the claims and specification are defined as set forth below unless otherwise specified.

The terms “subject” or “patient” are used interchangeably and encompass a cell, tissue, or organism, human, or non-human, whether in vivo, ex vivo, or in vitro, male, or female.

The term “mammal” encompasses both humans and non-humans and includes but is not limited to humans, non-human primates, canines, felines, murines, bovines, equines, and porcines.

The phrases “electronic records” and “electronic data” are used interchangeably and generally refer to patient data stored in electronic form. Examples of electronic records described herein include electronic health records and claims data.

The term “obtaining a dataset comprising electronic records of a patient” and variants thereof encompasses obtaining dataset comprising electronic records captured from the patient. Obtaining the dataset comprising electronic records can encompass performing steps of capturing the dataset e.g., obtaining data from the patient and recording the data. The phrase can also encompass receiving the dataset, e.g., from a third party that has performed the steps of capturing the dataset comprising electronic records from the patient. The term “obtaining a dataset comprising electronic records of a patient” can also include having (e.g., instructing) a third party obtain the dataset.

The term “sample” or “test sample” can include a single cell or multiple cells or fragments of cells or an aliquot of body fluid, such as a blood sample, taken from a patient, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour. In various embodiments, a sample can be a biopsy of a tissue, such as a lung tumor or a lung nodule.

The phrase “at risk for cancer” refers to a risk that a patient will develop cancer within a given time period, e.g., within 1 year. In various embodiments, the risk of cancer refers to a likelihood that a patient will develop cancer within a given time period from time zero (TO), wherein time zero refers to when electronic data was obtained from the patient. In various embodiments, the risk of cancer refers to a likelihood that a patient will develop cancer within a certain period, for example, 6 months, 1 year, 10 years, or 20 years.

The terms “treating,” “treatment,” or “therapy” of lung cancer shall mean slowing, stopping, or reversing a cancer's progression by administration of treatment. In some embodiments, treating lung cancer means reversing the cancer's progression, ideally to the point of eliminating the cancer itself. In various embodiments, “treating,” “treatment,” or “therapy” of lung cancer includes administering a therapeutic agent or pharmaceutical composition to the patient. Additionally, as used herein, “treating,” “treatment,” or “therapy” of lung cancer further includes administering a therapeutic agent or pharmaceutical composition for prophylactic purposes. Prophylaxis of a cancer refers to the administration of a composition or therapeutic agent to prevent the occurrence, development, onset, progression, or recurrence of cancer or some or all the symptoms of lung cancer or to lessen the likelihood of the onset of lung cancer.

It must be noted that, as used in the specification, the singular forms “a,” “an” and “the” include plural referents unless the context clearly dictates otherwise.

### II. SYSTEM ENVIRONMENT OVERVIEW

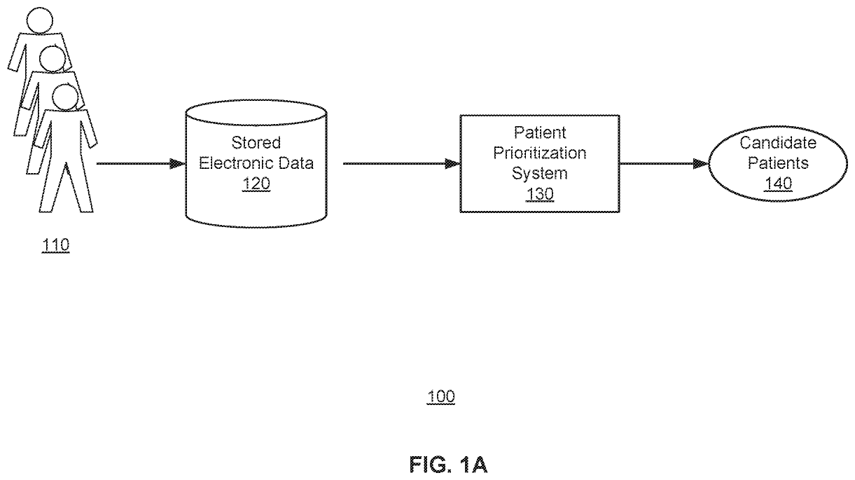

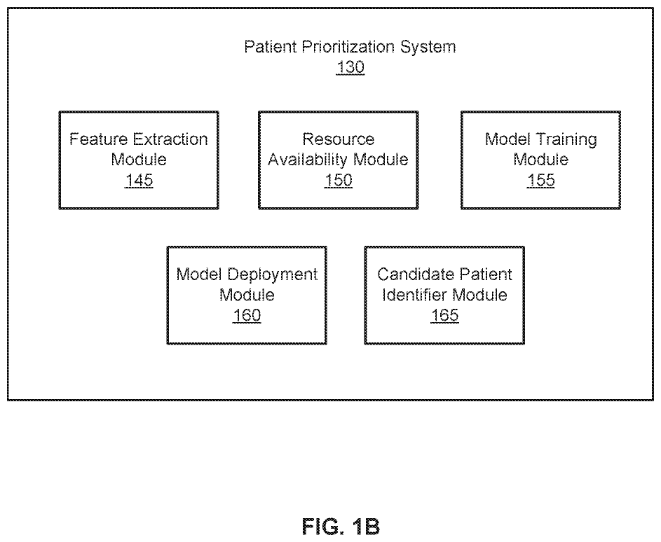

FIG. 1A depicts a system overview for prioritizing medical resources for candidate patients, in accordance with an embodiment. The system environment 100 provides context to introduce patients 110, stored electronic data 120, and a patient prioritization system 130 for identifying candidate patients 140. Although FIG. 1A depicts a system environment 100 including three patients 110, in various embodiments, the system environment 100 includes additional or fewer patients such that that patient prioritization system 130 identifies a subset of patients as candidate patients 140. For example, the system environment 100 may include 2, 100, 1000, 1 million, 100 million, or other number of patients.

In various embodiments, the patients 110 are presumed to be healthy. For example, the patients 110 have not been previously diagnosed with cancer. As another example, the patients 110 have not been previously suspected of having cancer. Thus, the methods disclosed herein can be beneficial for identifying candidate patients who may be at risk of cancer from patients who are presumed to be healthy. In various embodiments, the type of cancer is a lung cancer. Thus, the methods described herein can be beneficial for prioritizing candidate patients for early detection of lung cancer. In various embodiments, a patient 110 may have been previously diagnosed with a cancer. For example, the patient 110 can be in remission and therefore, the methods disclosed herein can be beneficial for determining whether the patient 110 is likely to experience a recurrence of cancer.

Generally, data can be obtained from the patients 110 and stored. For example, such data can include electronic data. FIG. 1A shows example stored electronic data 120 that is obtained from patients 110. Exemplary electronic data include electronic health record (EHR) data and claims data. EHR data represents an electronic version of a patient's medical history. Claims data includes administrative data covering information such as doctor's appointments, bills, and insurance information. Additional details and examples of EHR data and claims data are further described herein.

In various embodiments, the stored electronic data 120 can be gathered from patients 110 at one or more patient visits (e.g., patient visits to a medical provider). Thus, upon each patient visit, the stored electronic data 120 can be further augmented or supplemented by the information gathered at that patient visit. As referred to herein, the stored electronic data 120 represents a temporally diverse dataset of electronic records including electronic data recorded at various timepoints (e.g., at various timepoints when the patient visited). In various embodiments, the stored electronic data 120 is maintained and updated in real-time as additional information is gathered from a patient. For example, the stored electronic data 120 of various patients can be maintained in a cloud service and therefore, can be continuously updated as new or updated information of patients 110 are obtained. Database management system for storing electronic data can be any suitable system, for example, EPIC Healthcare Software, EBS PathoSof, HxRx Healthcare Management System, Healcon Practice, Drug Inventory Management System (DIMS), oeHealth, Patientpop, Webptis, GeBBS HIM Solutions, Cerner, WebPT, eClinicalWorks, and NextGen Healthcare EHR.

In various embodiments, the stored electronic data 120 and the patient prioritization system 130 are maintained or employed by a common party. In some embodiments, the stored electronic data 120 and the patient prioritization system 130 are maintained or employed by different parties. For example, a first party may maintain the stored electronic data 120 and/or continuously update the stored electronic data 120 in view of new or updated information from patients 110. A different party may operate the patient prioritization system 130 to analyze the stored electronic data 120 to identify candidate patients 140. In one example, the party that maintains the stored electronic data 120 may be a hospital or physician's office. Thus, the patient prioritization system 130 may request for and access the stored electronic data 120 maintained by a hospital or a physician's office to identify candidate patients 140.

Although FIG. 1A shows a single stored electronic data 120, in various embodiments there may be a plurality of stored electronic data 120. For example, each stored electronic data 120 can be maintained by a different party (e.g., a different hospital or physician's office) or multiple parties. Therefore, the patient prioritization system 130 can access and analyze stored electronic data 120 from a large number of patients by accessing stored electronic data 120 from various sources maintained by any number of parties.

Referring to the patient prioritization system 130, it accesses and analyzes stored electronic data 120 to identify candidate patients 140 that may be at risk of cancer. Candidate patients 140 may represent a subset of the patients 110. Candidate patients 140 may be prioritized to receive a subsequent intervention, whereas patients that are not identified as candidate patients 140 (patients not identified as candidate patients are hereafter referred to as “non-candidate patients”) may be deprioritized, in an embodiment.

In various embodiments, the patient prioritization system 130 accesses the stored electronic data 120 by sending a request to a party that maintains the stored electronic data 120. In various embodiments, the patient prioritization system 130 continuously accesses the stored electronic data 120 over time. Continuously accessing the stored electronic data 120 over time may enable the patient prioritization system 130 to access the most up-to-date stored electronic data 120 such that the patient prioritization system 130 can identify the candidate patients based on the most up-to-date stored electronic data 120. In various embodiments, the patient prioritization system 130 accesses the stored electronic data 120 at predetermined intervals of time (e.g., daily, bi-weekly, weekly, monthly, annually, or other suitable time intervals). In various embodiments, the party maintaining the stored electronic data 120 can provide the stored electronic data 120 to the patient prioritization system 130 when a trigger event occurs. For example, a trigger event may be an update to the stored electronic data 120 in view of new patient information or change to patient information.

In some embodiments the patient prioritization system 130 analyzes stored electronic data 120, such as EHR data, claims data, or other data that may be easily obtained and/or does not require extensive computing resource to analyze. Using easily obtainable data allows a larger pool of patients, making it suitable for use in early-stage cancer screening.

To identify candidate patients 140, the patient prioritization system 130 may analyze stored electronic data 120 of patients 110 by deploying a trained machine learning model, hereafter referred to as a trained risk prediction model. The trained risk prediction model may analyze features derived from the stored electronic data 120 of patients 110 to determine which patients are to be categorized as candidate patients 140.

Reference is now made to FIG. 1B, which depicts a block diagram of an example patient prioritization system 130. In this example, the patient prioritization system 130 includes a feature extraction module 145, a resource availability module 150, a model training module 155, a model deployment module 160, and a candidate patient identifier module 165. In various embodiments, the patient prioritization system 130 may be configured differently with additional, fewer, or different modules.

The feature extraction module 145 may process electronic data, such as electronic data obtained from stored electronic data 120 shown in FIG. 1A. In various embodiments, the feature extraction module 145 may identify eligible patients according to one or more criteria (e.g., 50-80 years old, non-smoker, and no prior scan, biopsy procedure, or cancer diagnosis). The feature extraction module 145 may extract features from the electronic data of eligible patients that meet the one or more criteria. In various embodiments, the feature extraction module 145 may assign weights to different features. For example, the feature extraction module 145 may assign higher weights to features from the electronic data 120 that were more recently recorded in comparison to features from the electronic data 120 that were earlier recorded in the electronic records. The feature extraction module 145 may provide the extracted features to the model deployment module 160, e.g., for inputting into a trained machine learning model.

The resource availability module 150 manages communications with one or more third parties to assess the availability of resources at the one or more third parties. Availability of resources at each third party may differ. For example, a third party may be a hospital or a physician's office that provides care to different numbers of patients. The resource availability module 150 may communicate with one or more third parties to receive indications identifying the quantity of available medical resources each third party has available. Based on the indications, the resource availability module 150 can determine whether the number of candidate patients identified for a particular third party exceeds the medical resources available at that third party. As an example, the resource availability module 150 may receive, from a third party, an indication that identifies that the third party has the capacity to perform X image scans for candidate patients. The resource availability module 150 can ensure that the total number of candidate patients identified to the third party does not exceed the third party's capacity of X image scans. In one embodiment, the resource availability module 150 sets a threshold according to the indication from the third party that reflects the available medical resources at the third party. By modulating the threshold, the resource availability module 150 can control the number of candidate patients identified for a particular third party.

The model training module 155 may perform steps to train one or more machine learning models. In some embodiments, the model training module 155 trains machine learning models such that the machine learning models can accurately separate candidate patients, who are likely at higher risk of developing cancer, from non-candidate patients, who are likely at lower risk of developing cancer. In some embodiments, the model training module 155 trains machine learning models using training data that includes electronic data, such as EHR data and/or claims data.

The model deployment module 160 may retrieve and deploy trained machine learning models to generate predictions for patients, the predictions being informative for determining whether a patient is categorized as a candidate patient or a non-candidate patient. In various embodiments, the model deployment module 160 deploys a trained machine learning model that outputs a score that is informative for determining whether a patient is categorized as a candidate patient or a non-candidate patient. In various embodiments, the trained machine earning model outputs relative contributions of feature groupings that contributed towards the score informative for determining whether a patient is categorized as a candidate patient or a non-candidate patient.

The candidate patient identifier module 165 may identify a subset of patients as candidate patients using the predictions generated by trained machine learning models. In various embodiments, to determine whether a patient is to be categorized as a candidate patient, the candidate patient identifier module 165 compares a prediction generated by the machine learning model for the patient to a threshold score. Based on the comparison, the candidate patient identifier module 165 may classify the patient as a candidate patient or a non-candidate patient. In some embodiments, the candidate patient identifier module 165 communicates with one or more third parties by providing identification of candidate patients. For example, the identified candidate patients may represent a subset of patients that are under the care of a third party. Thus, by providing identification of the candidate patients to the third party, the third party may then prioritize its available medical resources by providing interventions to the candidate patients over non-candidate patients.

### III. METHODS FOR PREDICTING RISK OF CANCER

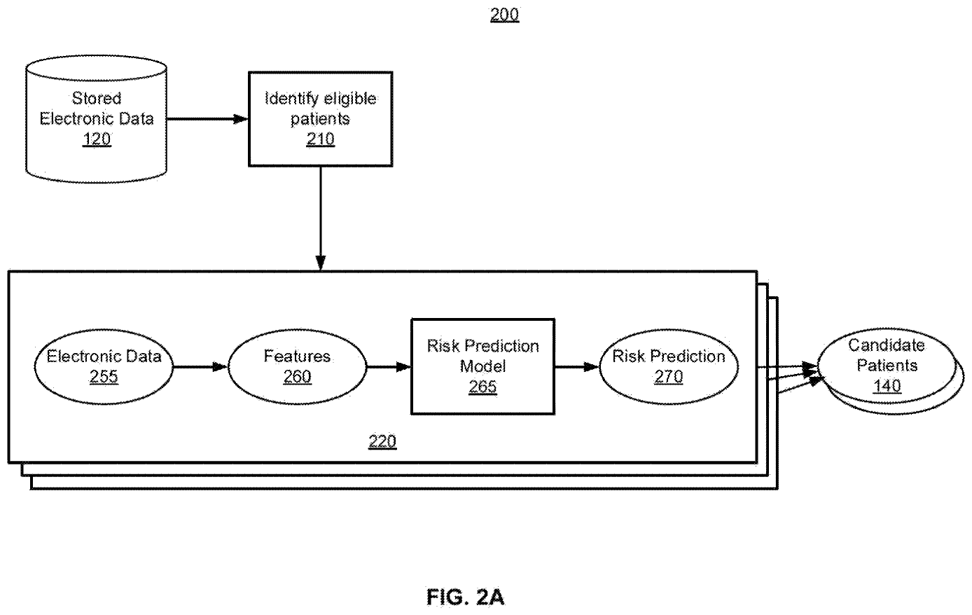

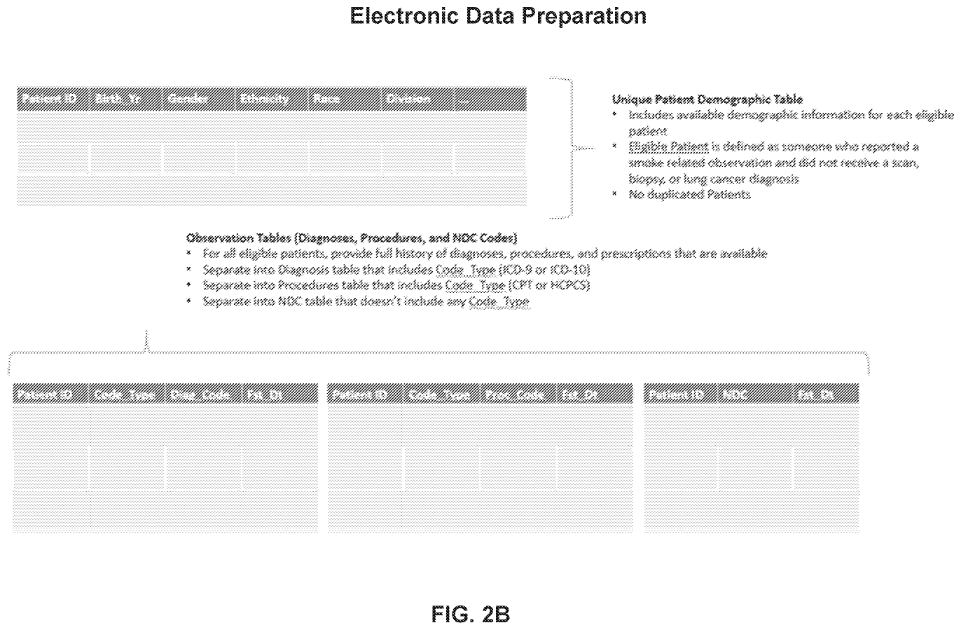

Embodiments described herein include methods for identifying candidate patients by applying one or more trained risk prediction models. Such methods can be performed by the patient prioritization system 130 described in FIG. 1B. Reference is now made to FIG. 2A, which depicts an example flow diagram for implementing a risk prediction model for identifying candidate patients.

In FIG. 2A, the patient prioritization system 130 receives or accesses the stored electronic data 120 (e.g., a temporally diverse dataset comprising electronic records of patients) that may be maintained by a third party. At step 210, the patient prioritization system 130 analyzes the stored electronic data 120 to identify eligible patients, for example, by identifying those satisfying one or more criteria. The one or more criteria can include a particular age group (e.g., between 30-100 years old, between 40-90 years old, or between 50-80 years old), smoking related observation (e.g., smoking habit of the patient, such as a smoking pack year history), and a lack of a prior scan, prior biopsy, or prior cancer diagnosis. Inclusion of the criteria of lacking a prior scan, prior biopsy, or prior cancer diagnosis may allow patients that may not typically be suspected of being at risk of cancer to be included as eligible patients. In various embodiments, the criteria may include one or more of the United States Preventive Services Task Force (USPSTF) recommendations, such as 50-80 years old and a 20+ smoking pack year history. In some embodiments, the criteria include each of age group (e.g., 50-80 years old), smoking related observation, and lack of each of a prior scan, prior biopsy, and prior cancer diagnosis.

In some embodiments, the patient prioritization system 130 identifies eligible patients by comparing the stored electronic data 120 of patients to the one or more criteria. If the electronic data 120 of a patient satisfies the criteria, the patient prioritization system 130 may identify the patient as an eligible patient and retain that patient's electronic data 120 for subsequent analysis. If the electronic data of a patient does not satisfy the criteria, in some embodiments the patient prioritization system 130 identifies the patient as an ineligible patient. The electronic data 120 of the ineligible patient may not be retained or may be set aside and not included for subsequent analysis.

In various embodiments, the patient prioritization system 130 organizes the electronic data 120 of the eligible patients prior to subsequent analysis. For example, the patient prioritization system 130 may organize the electronic data of eligible patients into one or more scalar tables to facilitate the subsequent analysis (e.g., to facilitate the later extraction of features). In various embodiments, different scalar tables can be generated for different types of electronic data. For example, a scalar table can be generated for EHR data and a second scalar table can be generated for claims data. As another example, separate scalar tables can be generated for patient demographic data, and observations data (e.g., diagnoses, procedures, and/or prescription codes). In some embodiments, four separate scalar tables are generated including 1) patient demographics table, 2) diagnosis table, 3) procedures table, and 4) prescriptions table.

Reference is now made to FIG. 2B, which depicts an example diagram for organizing the electronic data, in accordance with an embodiment. Specifically, the top of FIG. 2B shows a unique patient demographic table that organizes the patient demographic data of patients. For example, the patient demographic data can include the patient's birth year, patient's gender, patient's ethnicity, patient's race, patient's division, and the like. In various embodiments, the patient demographic table may only include demographic data of eligible patients. The bottom three tables shown in FIG. 2B include a diagnoses table, a procedures table, and a prescriptions table using for example, National Drug Code (NDC). The diagnoses table shown on the bottom left of FIG. 2B includes an identifier of the patient (e.g., “Patient ID”) and further includes codes for one or more diagnoses (shown as “Code_Type” and “Diag_Code”). Example code types include ICD-9 or ICD-10 codes. Example ICD-9 and ICD-10 codes are described in further detail herein. Furthermore, the diagnoses table can include a time (e.g., estimated date) at which the data was recorded (shown as “Est_Dt”). The procedures table shown in the bottom middle of FIG. 2B includes an identifier of the patient (e.g., “Patient ID”) and further includes codes for one or more procedures (shown as “Code_Type” and “Proc_Code”). Example code types include Current Procedural Terminology (CPT) or Healthcare Common Procedure Coding System (HCPCS) codes. Example CPT/HCPCS codes are described in further detail herein. Furthermore, the diagnoses table can include a time (e.g., estimated date) at which the data was recorded (shown as “Est_Dt”). The prescriptions (NDC) table shown in the bottom right of FIG. 2B includes an identifier of the patient (e.g., “Patient ID”) and further includes one or more prescriptions (shown as “NDC”). Example prescriptions are described in further detail herein. Furthermore, the diagnoses table can include a time (e.g., estimated date) at which the data was recorded (shown as “Est_Dt”).

Returning to FIG. 2A, for each of the eligible patients, a subsequent analysis 220 is performed to analyze the electronic data of the patient and to determine whether the patient is to be categorized as candidate patient or a non-candidate patient. As shown in FIG. 2A, the analysis 220 includes accessing the electronic data 255 of the patient, extracting features 260, and analyzing the extracted features using a risk prediction model 265 to generate a risk prediction 270. The risk prediction 270 is useful for determining whether the patient is to be categorized as candidate patient or a non-candidate patient. As shown in FIG. 2A, the analysis 220 can be performed multiple times for multiple patients. For example, each performance of the analysis 220 is patient specific. For example, for Z eligible patients, the analysis 220 can be performed Z times to determine whether the Z eligible patients are to be categorized as candidate patients or non-candidate patients.

A first step in the analysis 220 involves extracting features 260 from the electronic data 255 of a patient. Here, this step may be performed by the feature extraction module 145 described in FIG. 1B. In various embodiments, features include any of a patient demographic datapoint, a diagnosis code, a procedures code, a prescriptions code, or a combination thereof.

A patient demographic datapoint can be a value representing any of patient gender, patient age, patient behavior, patient ethnicity, patient race, patient geographic location, patient socioeconomic status, education status, number of months active, and/or insurance status. For example, a value representing the patient age can, in various embodiments, be the patient age itself. As another example, a value for a patient ethnicity may be an integer value, where a particular integer value represents a particular patient ethnicity.

A diagnosis code feature can be a diagnosis code, such as an ICD-9 or an ICD-10 diagnosis code (or information representative thereof). Example ICD-9 and ICD-10 codes are shown below in Tables 1 and 2. A procedures code feature can be a procedures code, such as a Current Procedural Terminology (CPT) or Healthcare Common Procedure Coding System (HCPCS) code (or information representative thereof). Example CPT and HCPCS codes are shown below in Tables 3 and 4. A prescriptions code feature can be a particular prescription. Example prescriptions are shown in Table 5.

In various embodiments, extracting the features 260 involves encoding the features that can then be analyzed by a machine learning model. For example, encoding the features can involve encoding the features into an input vector that can be analyzed by a machine learning model. Any suitable way to encode categorical variables may be used to encode the features, for example, one-hot encoding, label/ordinal encoding, target encoding, feature hashing, binary encoding, or count encoding.

In various embodiments, the feature extraction module 145 may differentially weigh the extracted features before inputting into the machine learning model. In some embodiments, differential weighing of the extracted features need not be performed. In some embodiments, the feature extraction module 145 differentially weighs the extracted features according to timepoints that the data were recorded. For example, the feature extraction module 145 assigns higher weights to features from the electronic data that were more recently recorded in comparison to features from data that were earlier recorded in the electronic record. Given that more recently recorded electronic data 120 may be more informative of the patient's current risk for cancer as opposed to electronic data 120 that was recorded earlier, the more recently recorded electronic is assigned a higher weight to reflect its increased informativeness. In some embodiments, weights may be assigned based on, but not limited to, dates, file sizes, medical professional names, and the like.

In various embodiments, differentially weighing the extracted features involves modifying the features according to different weight values. For example, if the features are encoded as an input vector, differentially weighing the features can involve modifying individual entries of the input vector by the different assigned weights to generate weighted features. Thus, values of features that are assigned higher weights can be increased relative to values of features that are assigned smaller weights.

The next step of the analysis 220 involves applying a risk prediction model 265 (e.g., by the model deployment module 160 shown in FIG. 1B) to analyze the features. In various embodiments, a risk prediction model analyzes the features and generates a risk prediction 270, which may be informative for determining whether the patient is to be categorized as a candidate patient or non-candidate patient. In various embodiments, the risk prediction 270 can be represented by a value. In various embodiments, the risk prediction 270 is a binary value (e.g., 0 or 1, where 0 indicates unlikely to develop cancer in a certain time period and 1 indicates likely to develop cancer in a certain time period). In various embodiments, the risk prediction 270 is represented by a score, such as a continuous value (e.g., between 0 and 1, where a value closer to 1 indicates higher likelihood of developing cancer).

In various embodiments, the risk prediction model 265 is a regression model (e.g., a logistic regression or linear regression model) that calculates a risk prediction 270 by combining a set of trained parameters with the extracted features. As another example, the risk prediction model can be a neural network model that calculates a risk prediction 270 by combining a set of trained parameters associated with nodes and layers of the neural network with values of the extracted features. As another example, the risk prediction model 265 can be a random forest model that calculates a risk prediction 270 by combining a set of trained parameters associated with decision tree nodes with values of the extracted features. As another example, the risk prediction model 265 can be a gradient boosted machine model that calculates a risk prediction 270 by combining a set of trained parameters associated with decision tree nodes with values of the extracted features.

In various embodiments, the risk prediction model 265 analyzes feature groupings, where a feature grouping represents 2 or more extracted features. Extracted features in a feature grouping may be related. For example, extracted features of a feature grouping can be related according to an anatomical organ or according to the patient, examples of which include patient behavior, patient characteristics, smoking status, and vaccination status. Exemplary feature groupings are described herein and further shown below in Table 6. In such embodiments, the risk prediction model 265 may combine individual features into respective feature groupings, and then analyzes the feature groupings to determine the risk prediction 270. In various embodiments, the risk prediction model 265 analyzes both individual features, as well as feature groupings in generating the risk prediction 270. In various embodiments, the risk prediction model 265 analyzes only feature groupings in generating the risk prediction 270.

In various embodiments, the risk prediction model 265 further performs an analysis to determine the relative contributions of feature groupings that resulted in the risk prediction 270. The relative contributions of feature groupings are additionally referenced herein as subscores. In various embodiments, the risk prediction model 265 determines a relative contribution of a features or feature grouping by constructing and calculating outputs across various scenarios, a subset of scenarios including the extracted feature or feature grouping and another subset of scenarios excluding the extracted feature or feature grouping. Thus, by analyzing the changes of the outputs across the various scenarios, the relative contribution of the extracted feature or feature grouping can be deduced.

Grouping features into feature groupings and performing analysis using feature grouping to determine relative contribution may use less resources than determining relative contribution using ungrouped features. In various embodiments, the risk prediction model 265 groups features into any number of feature groupings such that the risk prediction model 265 only needs to determine a reduced number of relative contributions.

In various embodiments, the risk prediction model 265 performs a Shapley Additive Explanation (SHAP) analysis to determine SHAP values for feature groupings. Generally, the SHAP analysis takes into account all different combinations of input variables with different subsets of the predictor vector as contributing to the output prediction. In various embodiments, the risk prediction model 265 performs a Kernel SHAP analysis that calculates contributions of feature groupings across fewer scenarios. For example, and without limitation, Kernel SHAP uses a weighted linear regression, where the coefficients of the linear regression represent the contributions of the feature groupings. The various scenarios may be used to determine weights of the linear regression to determine the contributions of the feature groupings. In some embodiments, other machine learning models and/or algorithms may be used to determine contributions of feature groupings, without limitation, such as supervised, unsupervised, or other machine learning models.

The risk prediction 270 may be calculated based on the combination of contributions from feature groupings. For example, the risk prediction 270 may, in various embodiments, be a summation of the contributions from individual feature groupings.

Given the risk prediction 270, the candidate patient identifier module 165, as shown in FIG. 1B, can compare the risk prediction 270 to a threshold value to determine whether the patient is a candidate patient or a non-candidate patient. The threshold value maybe a fixed score. For example, if the risk prediction 270 is above the threshold score, the patient is classified into one category (e.g., a candidate patient). Alternatively, if the risk prediction 270 is below the threshold score, the patient is classified into a different category (e.g., non-candidate patient).

In various embodiments, the threshold score is set according to a quantity of available medical resources at a third party. Thus, each threshold score may represent a custom threshold score that is personalized for each third party. For example, as described herein, a third party may provide an indication that reflects the available medical resources of the third party. Thus, the candidate patient identifier module 165 may set a threshold score according to the indication that reflects the available medical resources. For example, for a third party that is severely limited on resources, the third party sends an indication reflecting those limited resources. The candidate patient identifier module 165 may set a high threshold value (e.g., at least 0.6, at least 0.7, or at least 0.8) such that fewer patients have scores above the threshold value and are identified as candidate patients. As another example, for a third party that is not limited on resources, the third party sends an indication reflecting non-limited resources. The candidate patient identifier module 165 may set a lower threshold value (e.g., at most 0.4, at most 0.3, or at most 0.2) such that more patients have scores above the threshold value and are identified as candidate patients. Threshold values may be set based on patient categories, classifications, demographics, diagnosis, and the like.

Following identification of the candidate patients 140, the patient prioritization system 130 provides the identification of the candidate patients 140 to a third party (e.g., a third party managing the care for the candidate patients). Thus, the third party can appropriately prioritize its available medical resources to provide interventions to the candidate patients.

In various embodiments, the patient prioritization system 130 can additionally provide, to the third party, identification of one or more feature groupings that contributed to the categorization of patients as candidate patients. Here, the patient prioritization system 130 can provide identification of feature groupings on a per-patient basis. For example, for each candidate patient, the patient prioritization system 130 provides identification of the specific feature groupings that contributed to the categorization of the patient as a candidate patient.

In various embodiments, the patient prioritization system 130 ranks the features or feature groupings that contributed to the categorization of a patient as a candidate patient. The patient prioritization system 130 can provide the top-ranked feature or top-ranked feature grouping. In various embodiments, the patient prioritization system 130 can provide one or more features or feature groupings in accordance with their rank. For example, the patient prioritization system 130 provides the top 3 features or feature groupings that contributed to the categorization of a patient as a candidate patient. For another example, the patient prioritization system 130 provides the third-ranked feature or feature grouping that contributed to the categorization of a patient as a candidate patient.

The third party can use the identification of features or feature groupings to select and provide care to a candidate patient. For example, if the top feature grouping that most heavily contribute to the patient being categorized as a candidate patient is the smoking behavior of the patient, the third party can appropriately counsel the candidate patient regarding the smoking behavior (e.g., counsel to reduce smoking or terminate smoking).

### IV. TRAINING A MACHINE LEARNING MODEL

Generally, a machine learning model is structured such that it analyzes features extracted from electronic data, such as features extracted from EHR data and/or features extracted from claims data, and generates a prediction informative for classifying a patient as a candidate or non-candidate patient.

The risk prediction model can use any suitable machine learning model, such as a regression model (e.g., linear regression, logistic regression, or polynomial regression), decision tree, random forest, gradient boosting (e.g., a XGBoost gradient boosting model or a CatBoost gradient boosting model), support vector machine, Naïve Bayes model, k-means cluster, or neural network (e.g., feed-forward networks, convolutional neural networks (CNN), deep neural networks (DNN), autoencoder neural networks, or any combination thereof).

The risk prediction model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the risk prediction model is trained using supervised learning algorithms, unsupervised learning algorithms, semi-supervised learning algorithms (e.g., partial supervision), weak supervision, transfer, multi-task learning, or any combination thereof.

In various embodiments, the risk prediction model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, node values in a decision tree, and coefficients in a regression model. The model parameters of the risk prediction model are trained (e.g., adjusted) using the training data to improve the predictive capacity of the risk prediction model.

The model training module 155 trains the risk prediction model using training data. In various embodiments, the training data includes extracted features from electronic data (e.g., EHR data and/or claims data) obtained from training individuals. As used herein, a training individual may be an individual known to be at risk or not be at risk for cancer. In various embodiments, a training individual may be an individual known to not be at risk for cancer if the training individual is not subsequently diagnosed with cancer. For example, such a training individual known to not be at risk for cancer may be an individual who later underwent an intervention (e.g., CT/PET imaging and/or biopsy) and was determined to not have cancer. In various embodiments, a training individual may be an individual known to be at risk for cancer if the training individual is subsequently diagnosed with cancer. For example, such a training individual known to be at risk for cancer may be an individual who later underwent an intervention (e.g., CT/PET imaging and/or biopsy) and was determined to have cancer. In various embodiments, a training individual may be an individual known to be at risk for cancer if the training individual is subsequently diagnosed with cancer at a timepoint at least X months in the future. For example, the at least X months in the future is sufficiently distant in the future such that when the electronic records were obtained from the training individual, the cancer in the training individual represented an early-stage cancer. In various embodiments, X may be at least 1 month. In some embodiments, X may be between about 1 month to about 55 months.

In various embodiments, the training data can be obtained from a split of a dataset. For example, the dataset can undergo a 50:50 training:testing dataset split. In some embodiments, the dataset can undergo a 60:40 training:testing dataset split. In some embodiments, the dataset can undergo a 80:20 training:testing dataset split.

In various embodiments, the training data used for training the imputation model includes reference ground truths that indicate that a training individual was subsequently diagnosed with cancer (hereafter also referred to as “positive” or “+”) or whether the training individual was not subsequently diagnosed with cancer (hereafter also referred to as “negative” or “−”). In various embodiments, the reference ground truths in the training data are binary values, such as “1” or “0.” For example, a training individual that was subsequently diagnosed with cancer can be identified in the training data with a value of “1” whereas a training individual who was not diagnosed with cancer can be identified in the training data with a value of “0.” In various embodiments, the model training module 155 trains the risk prediction model using the training data to minimize a loss function such that the risk prediction model can better generate a prediction (e.g., a score informative for determining whether the patient is a candidate or non-candidate patient) based on the input (e.g., extracted features of the electronic data). In various embodiments, the loss function is constructed for any of a least absolute shrinkage and selection operator (LASSO) regression, Ridge regression, or ElasticNet regression.

In various embodiments, risk prediction models disclosed herein achieve a performance metric. Example performance metrics include an area under the curve (AUC) of a receiver operating curve, a positive predictive value, and/or a negative predictive value. In various embodiments, risk prediction models disclosed herein exhibit an AUC value of at least 0.5. In various embodiments, risk prediction models disclosed herein exhibit an AUC value of at least 0.6. In various embodiments, risk prediction models disclosed herein exhibit an AUC value of at least 0.7. In various embodiments, risk prediction models disclosed herein exhibit an AUC value of at least 0.8. In various embodiments, risk prediction models disclosed herein exhibit an AUC value of at least 0.9. In various embodiments, risk prediction models disclosed herein exhibit an AUC value of at least 0.95. In various embodiments, risk prediction models disclosed herein exhibit an AUC value of at least 0.99. In various embodiments, risk prediction models disclosed herein exhibit an AUC value of at least 0.51. In some embodiments, AUC values may be between about 0.51 to about 0.99, without limitation.

In various embodiments, risk prediction models disclosed herein achieve an odds ratio, which refers to the relative risk of the higher risk population (e.g., candidate patients) compared to the standard risk. In various embodiments, risk prediction models disclosed herein achieve an odds ratio of at least 1.1 to about 3.0, without limitation.

### V. EXAMPLE METHODS FOR PRIORITIZATION MEDICAL RESOURCES FOR SCREENING CANCER PATIENTS

As described herein, example methods for prioritizing medical resources for screening cancer patients involve analysis of features from electronic records using a trained machine learning model. In some embodiments, the features of the electronic records are weighted according to timepoints that data were received. For example, more recently recorded data in the electronic records can be assigned a higher weight than earlier recorded data in the electronic records. More recently recorded data may be more reflective of a current state of the patient and therefore, a higher weight of the more recently recorded data enables the machine learning model to appropriately account for the timing of the recordation. Altogether, this enables the machine learning model to predict candidate patients more accurately.

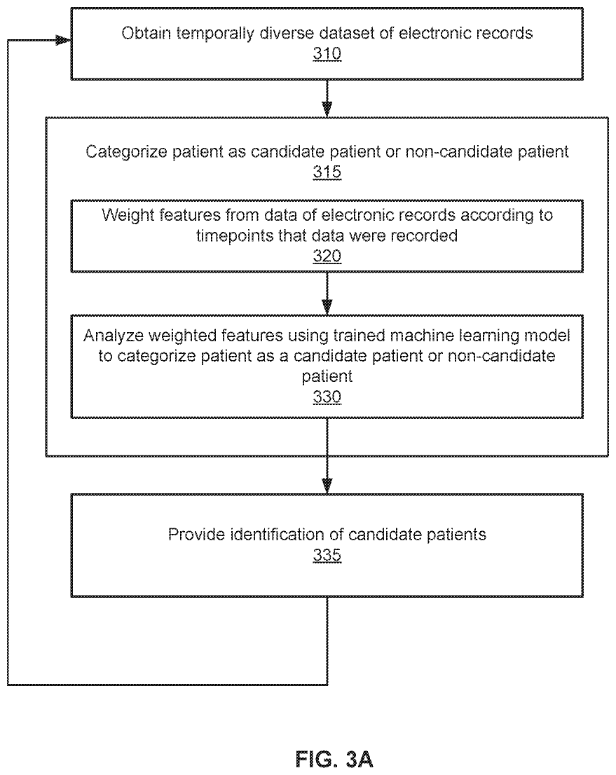

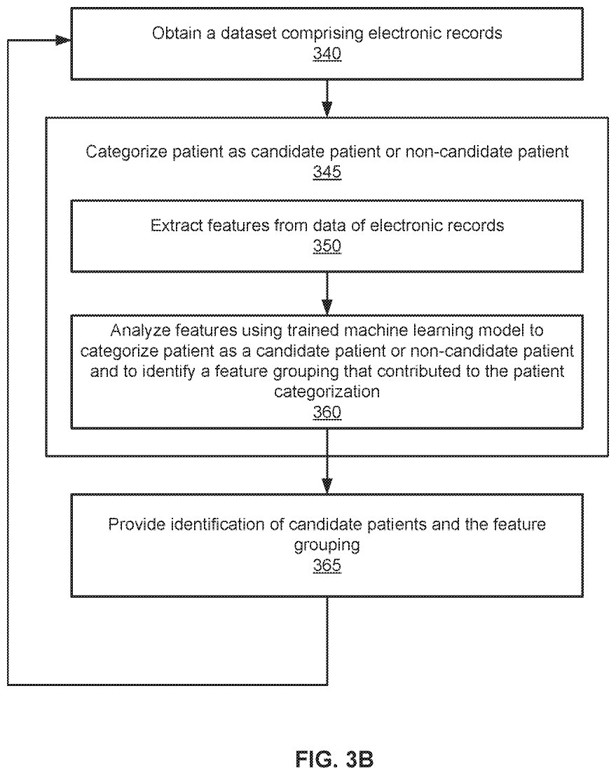

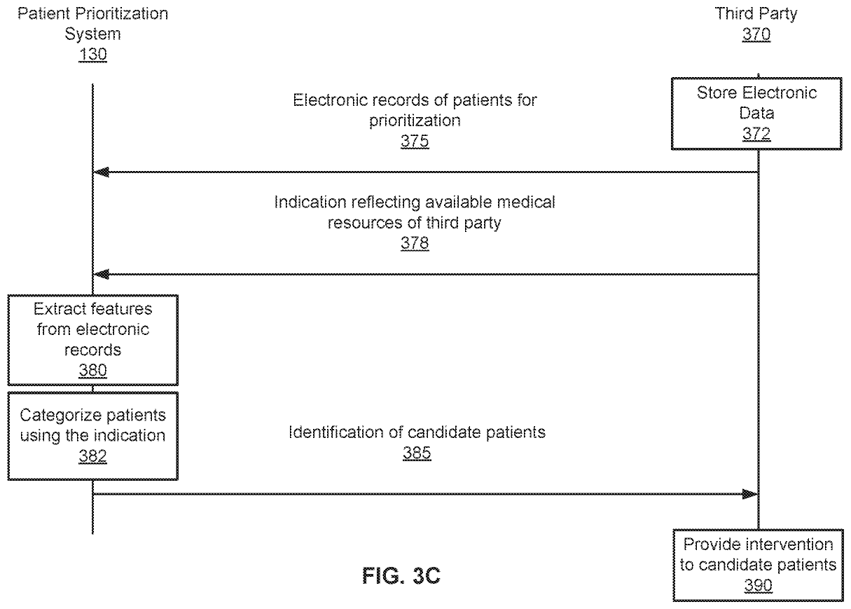

Reference is now made to FIG. 3A, which depicts an example flow process for identifying candidate patients, in accordance with a first embodiment. Step 310 involves obtaining a temporally diverse dataset of electronic records. Here, the temporally diverse dataset includes information of patients that are recorded at various timepoints. For example, for a patient, the temporally dataset may include EHR and/or claims data obtained from the patient during a first hospital visit, and may further include EHR and/or claims data obtained from the same patient during a second hospital visit.

Step 315 involves an overall step of categorizing a patient as a candidate patient or a non-candidate patient. As shown in FIG. 3A, step 315 includes step 320 and step 330. Step 315 can be performed multiple times across different patients to determine whether each of the patients are a candidate patient or a non-candidate patient.

Step 320 involves weighting features from data of the electronic records according to timepoints that the data of the electronic records were recorded. Specifically, features from data more recently recorded in the electronic records are more heavily weighted in compared to features from data that were earlier recorded in the electronic records.

Step 330 involves analyzing the weighted features using a trained machine learning model. The trained machine learning model outputs a prediction for categorizing the patient as a candidate patient or a non-candidate patient.

Step 335 involves providing identification of the candidate patients. In various embodiments, step 335 involves providing identification of the candidate patients to a third party that is managing the care of the patient (e.g., a hospital or physician's office).

As shown in FIG. 3A, the flow process can restart again at step 310. Here, a new temporally diverse dataset of electronic records can be obtained. Here, the temporally diverse dataset of electronic records may include data that was newly recorded since a prior version of the temporally diverse dataset was obtained.

In various embodiments, example methods for prioritizing medical resources for screening cancer patients involve analyze groupings of features from electronic records using a trained machine learning model. Here, these feature groupings can be analyzed to determine how much each feature grouping contributed to the prediction of the machine learning model (e.g., a prediction informative of a candidate patient or a non-candidate patient).

Reference is now made to FIG. 3B, which depicts an example flow process for identifying candidate patients, in accordance with a second embodiment. Step 340 involves obtaining a dataset comprising electronic records. The electronic records may include one or both of EHR data and claims data for one or more patients.

Step 345 involves an overall step of categorizing a patient as a candidate patient or a non-candidate patient. As shown in FIG. 3B, step 345 includes step 350 and step 360. Step 345 can be performed multiple times across different patients to determine whether each of the patients are a candidate patient or a non-candidate patient.

Step 350 involves extracting features from data of the electronic records. Example features from EHR data and/or claims data are described herein.

Step 360 involves analyzing features using a trained machine learning model. The machine learning model can output a score indicative of cancer risk for the patient. The score indicative of cancer risk is determinative of whether the patient is a candidate patient or a non-candidate patient. In various embodiments, the machine learning model can further identify a feature grouping that contributed to the score indicative of cancer risk. For example, the identified feature grouping may be a feature grouping that most heavily contributed to the score indicative of cancer risk.

Step 365 involves providing identification of the candidate patients and the corresponding identifications of feature groupings. In various embodiments, step 365 involves providing identification of the candidate patients and the corresponding identifications of feature groupings to a third party that is managing the care of the patient (e.g., a hospital or physician's office).

As shown in FIG. 3B, the flow process can restart again at step 340. Here, a new dataset of electronic records can be obtained. The new dataset of electronic records may include data that was newly recorded since a prior version of the temporally diverse dataset was obtained.

In various embodiments, example methods for prioritizing medical resources for screening cancer patients involve categorizing patients according to an indication reflecting available medical resources of a third party. For example, the third party may manage the care of various patients and may gave limited medical resources. Thus, the third party may need to prioritize the medical resources for a subset of the patients (e.g., candidate patients). An example third party may be a hospital or physician's office that cares for the patients and/or stores electronic records (e.g., EHR data and/or claims data) related to the patients. Methods disclosed herein can be useful for identifying candidate patients such that the third party can prioritize medical resources and provide interventions to the candidate patients first.

Reference is now made to FIG. 3C, which depicts an example interaction diagram for identifying candidate patients, in accordance with a third embodiment. The interaction diagram shows an example patient prioritization system 130 and a third party 370. At step 372, the third-party stores electronic data, such as electronic data for one or more patients. At step 375, the patient prioritization system 130 receives the electronic data of patients. Furthermore, at step 378, the patient prioritization system 130 receives an indication reflecting the available medical resources of the third party 370. At step 380, the patient prioritization system 130 extracts features from data of the electronic records (e.g., EHR data and/or claims data). Example features of EHR data and/or claims data is further described herein.

At step 382, the patient prioritization system 130 categorizes patients using the indication received from the third party 370. Here, step 382 involves analyzing features using a trained machine learning model to generate a prediction of whether patients are to be categorized as candidate patients or non-candidate patients. In some embodiments, the patient prioritization system 130 establishes a threshold score using the indication received from the third party 370 such that the machine learning model uses the threshold score to categorize patients as candidate patients or non-candidate patients. The patient prioritization system 130 sets a threshold score to meet the available medical resources for the third party 370. Thus, at step 385, the patient prioritization system 130 can provide identification of a tailored set of candidate patients to the third party 370. Then, at step 390, the third party 370 can provide an intervention to the candidate patients, while withholding the intervention for non-candidate patients.

### VI. EXAMPLE ELECTRONIC DATA

Methods disclosed herein involve analyzing electronic data of patients to categorize patients as candidate or non-candidate patients. Electronic data generally refers to data gathered from patients that are stored in electronic form. Exemplary electronic data include electronic health record (EHR) data and claims data of patients. In various embodiments, electronic data further includes timepoints for which the EHR data and/or claims data of patients were recorded.

Referring first to EHR data, it represents readily available medical information that may have been previously obtained from patients (e.g., obtained over one or more patients to a hospital or physician's office). For example, EHR data represents an electronic version of a patient's medical history. In various embodiments, EHR data comprises one or more of patient demographics data, laboratory test data, prior diagnoses data, prior procedures data, and prior prescriptions data. In some embodiments, EHR data comprises each of patient demographics data, prior diagnoses data, prior procedures data, and prior prescriptions data.

Referring to claims data, it represents administrative data collected from patients, examples of which include information from doctor's appointments, bills, and insurance information. In various embodiments, claims data comprise one or more of patient demographics data, prior diagnoses data, prior procedures data, and prior prescriptions data. In various embodiments, claims data comprise each of patient demographics data, prior diagnoses data, prior procedures data, and prior prescriptions data.

Patient demographics data of the EHR data and/or the claims data can refer to background characteristics of the patient. Example patient demographics data include patient gender, patient age, patient behavior, patient ethnicity, patient race, patient geographic location, patient socioeconomic status, education status, number of months active (e.g., number of months for which EHR data was stored for a patient), and/or insurance status. In some embodiments, patient demographics data includes patient behavior. Examples of patient behavior can include number of prior hospitalizations, number of prior physician visits, number of emergency room visits, and number of unique providers. In some embodiments, the patient behavior includes the smoking behavior of a patient. The smoking behavior of a patient can be identified as one of current smoker, previously smoked, never smoked, other smoking, not currently smoking, or unknown smoker.

Prior diagnoses data of the EHR data and/or the claim data can refer to a number of prior diagnoses and/or identifications of prior diagnoses for the patient. Such diagnoses data of the EHR data can include diagnosis codes, which correspond to diagnoses of lung-related or non-lung related issues. Example diagnoses codes for diagnosing lung-related issues are shown below in Table 1. Furthermore, example diagnoses codes for identifying diagnosing non-lung related issues are shown below in Table 2.

TABLE 1
Example Diagnosis Codes for Identifying Lung-Related Issues

| ICD-10 | ICD-9 | |
|---|---|---|
| Code | Code | Description |
| J449 | 49120 | Chronic obstructive pulmonary disease, unspecified |
| J440 | 49122 | Chronic obstructive pulmonary disease with (acute) lower respiratory infection |
| J441 | 49121 | Chronic obstructive pulmonary disease with (acute) exacerbation |
| J209 | 4660 | Acute bronchitis, unspecified |
| J410 | 4910 | Simple chronic bronchitis |
| J411 | 4911 | Mucopurulent chronic bronchitis |
| J418 | 4918 | Mixed simple and mucopurulent chronic bronchitis |
| J42 | 4919 | Unspecified chronic bronchitis |
| J439 | 4920 | Emphysema, unspecified |
| J181 | 481 | Lobar pneumonia, unspecified organism |
| J189 | 486 | Pneumonia, unspecified organism |
| J4540 | 49300 | Moderate persistent asthma, uncomplicated |
| J45909 | 49390 | Unspecified asthma, uncomplicated |
| J90 | 5111 | Pleural effusion, not elsewhere classified |
| J9601 | 51851 | Acute respiratory failure with hypoxia |
| J9600 | 51851 | Acute respiratory failure, unspecified whether hypoxia or hypercapnia |
| J9602 | 51851 | Acute respiratory failure with hypercapnia |
| J9611 | 51883 | Chronic respiratory failure with hypoxia |
| J9621 | 51853 | Acute and chronic respiratory failure with hypoxia |
| J9622 | 51853 | Acute and chronic respiratory failure with hypercapnia |
| J984 | 51889 | Other disorders of lung |
| R05 | 7862 | Cough |
| R0600 | 78609 | Dyspnea, unspecified |
| R062 | 78607 | Wheezing |
| R600 | 7823 | Localized edema |
| Z7951 | V5865 | Long term (current) use of inhaled steroids |
| Z9981 | V462 | Dependence on supplemental oxygen |
| R079 | 78650 | Chest pain, unspecified |
| I272 | 4168 | Other secondary pulmonary hypertension |
| I2720 | 4168 | Pulmonary hypertension, unspecified |
| I739 | 4439 | Peripheral vascular disease, unspecified |
| I7389 | 44389 | Other specified peripheral vascular diseases |
| I70219 | 44021 | Atherosclerosis of native arteries of extremities with intermittent claudication, |
| | | unspecified extremity |
| I70213 | 44021 | Atherosclerosis of native arteries of extremities with intermittent claudication, |
| | | bilateral legs |
| I701 | 4401 | Atherosclerosis of renal artery |
| I700 | 4400 | Atherosclerosis of aorta |
| I2510 | 41400 | Atherosclerotic heart disease of native coronary artery without angina pectoris |
| I6523 | 43310 | Occlusion and stenosis of bilateral carotid arteries |
| I6521 | 43310 | Occlusion and stenosis of right carotid artery |
| I6522 | 43310 | Occlusion and stenosis of left carotid artery |
| I6529 | 43310 | Occlusion and stenosis of unspecified carotid artery |
| I110 | 40201 | Hypertensive heart disease with heart failure |
| I252 | 412 | Old myocardial infarction |
| I5032 | 42832 | Chronic diastolic (congestive) heart failure |
| I5022 | 42822 | Chronic systolic (congestive) heart failure |
| E1151 | 25070 | Type 2 diabetes mellitus with diabetic peripheral angiopathy without gangrene |
| E1142 | 3572 | Type 2 diabetes mellitus with diabetic polyneuropathy |
| E119 | 25000 | Type 2 diabetes mellitus without complications |
| E871 | 2761 | Hypo-osmolality and hyponatremia |
| R0902 | 79902 | Hypoxemia |
| E782 | 2722 | Mixed hyperlipidemia |
| E785 | 2724 | Hyperlipidemia, unspecified |
| D508 | 2801 | Other iron deficiency anemias |
| I10 | 4010 | Essential (primary) hypertension |
| E7800 | 2720 | Pure hypercholesterolemia, unspecified |
| D649 | 2859 | Anemia, unspecified |
| I480 | 42731 | Paroxysmal atrial fibrillation |
| Z950 | V4501 | Presence of cardiac pacemaker |
| Z952 | V433 | Presence of prosthetic heart valve |
| Z951 | V4581 | Presence of aortocoronary bypass graft |
| Z955 | V4582 | Presence of coronary angioplasty implant and graft |
| Z961 | V431 | Presence of intraocular lens |
| H2512 | 36616 | Age-related nuclear cataract, left eye |
| H2511 | 36616 | Age-related nuclear cataract, right eye |
| H2513 | 36616 | Age-related nuclear cataract, bilateral |
| E440 | 2630 | Moderate protein-calorie malnutrition |
| K922 | 5789 | Gastrointestinal hemorrhage, unspecified |
| K921 | 5781 | Melena |
| K219 | 53081 | Gastro-esophageal reflux disease without esophagitis |
| Z681 | V850 | Body mass index (BMI) 19.9 or less, adult |
| Z923 | V153 | Personal history of irradiation |
| E43 | 261 | Unspecified severe protein-calorie malnutrition |
| D500 | 2800 | Iron deficiency anemia secondary to blood loss (chronic) |
| E875 | 2767 | Hyperkalemia |
| G894 | 3384 | Chronic pain syndrome |
| R0602 | 78605 | Shortness of breath |
| Z7982 | V5866 | Long term (current) use of aspirin |
| Z7901 | V5861 | Long term (current) use of anticoagulants |
| Z79891 | V5869 | Long term (current) use of opiate analgesic |
| D509 | 2809 | Iron deficiency anemia, unspecified |
| M1990 | 71530 | Unspecified osteoarthritis, unspecified site |
| R490 | 78442 | Dysphonia |
| E538 | 2662 | Deficiency of other specified B group vitamins |
| G8929 | 33829 | Other chronic pain |
| E559 | 2689 | Vitamin D deficiency, unspecified |
| F1120 | 30400 | Opioid dependence, uncomplicated |
| M069 | 7140 | Rheumatoid arthritis, unspecified |
| F17290 | 3051 | Nicotine dependence, other tobacco product, uncomplicated |
| Z720 | V698 | Tobacco use |
| F17200 | 3051 | Nicotine dependence, unspecified, uncomplicated |
| F17210 | 3051 | Nicotine dependence, cigarettes, uncomplicated |
| Z87891 | V1582 | Personal history of nicotine dependence |

TABLE 2
Example Diagnosis Codes for Identifying Non-Lung Related Issues

| ICD-10 | ICD-9 | |
|---|---|---|
| Code | Code | Description |
| F489 | 3009 | Nonpsychotic mental disorder, unspecified |
| Z6843 | V8543 | Body mass index (BMI) 50.0-59.9, adult |
| Z01419 | V7231 | Encounter for gynecological examination (general) (routine) without |
| | | abnormal findings |
| E291 | 2572 | Testicular hypofunction |
| Z01411 | V7231 | Routine gynecological examination |
| J029 | 462 | Acute pharyngitis |
| Z131 | V771 | Screening for diabetes mellitus |
| R030 | 7962 | Elevated blood pressure reading without diagnosis of hypertension |
| E6601 | 27801 | Morbid obesity |

Prior procedures data of the EHR data and/or the claim data can refer to a number of prior procedures and/or identifications of prior procedures for the patient. Such procedures data can include procedure codes, which correspond to performed procedures for lung-related or non-lung related issues. Example procedure codes for lung-related procedures are shown below in Table 3. Furthermore, example procedure codes for non-lung related procedures are shown below in Table 4.

TABLE 3
Example Procedure Codes for Lung-related procedures

| Proc | | |
|---|---|---|
| Code | Code Type | Description |
| 99406 | CPT/HCPCS | Behavior change smoking 3-10 min |
| 3077F | CPT/HCPCS | Systolic blood pressure >/=140 mm hg |
| 3078F | CPT/HCPCS | Diastolic blood pressure <80 mm hg |
| 94060 | CPT/HCPCS | Evaluation of wheezing |
| 94640 | CPT/HCPCS | Airway inhalation treatment |
| 84520 | CPT/HCPCS | Assay of urea nitrogen |
| 99223 | CPT/HCPCS | Initial hospital care |
| J1644 | CPT/HCPCS | Injected heparin sodium per 1000 u |
| 99232 | CPT/HCPCS | Subsequent hospital care |

TABLE 4
Example Procedure Codes for Non-Lung related procedures

| Proc | | |
|---|---|---|
| Code | Code Type | Description |
| G8484 | CPT/HCPCS | Flu immunize no admin |
| G8752 | CPT/HCPCS | Sys bp less 140 |
| G8754 | CPT/HCPCS | Dias bp less 90 |
| 80050 | CPT/HCPCS | General health panel |
| 99396 | CPT/HCPCS | Previous visit estimated age 40-64 |
| 99386 | CPT/HCPCS | Previous visit new age 40-64 |
| 1036F | CPT/HCPCS | Tobacco non-user |
| 3017F | CPT/HCPCS | Colorectal cancer screening |
| | | documented and reviewed |

Prior prescriptions data of the EHR data and/or the claim data can refer to a number of prior prescriptions and/or identifications of prior prescriptions that were provided to the patient. Example prior prescriptions can include prescriptions for treating a lung-related condition or a non-lung related condition. Example prior prescriptions are shown below in Table 5. The right-most column of Table 5 shows the target body area of the drug, including lung and non-lung (e.g., blood, digestion, heart, mental, allergies, skin, reproductive, hormone, smoke, vaccine, and general) conditions.

TABLE 5
Example Prescriptions

| Prescription | Description | Body Area |
|---|---|---|
| Tiotropium Bromide | Treats lung diseases - COPD or Asthma | Lung |
| Albuterol Sulfate | Bronchodilator used to treat or prevent | Lung |
| | bronchospasm | |
| Fluticasone Propion/ | Treat difficulty breathing, wheezing, | Lung |
| Salmeterol | shortness of breath | |
| Lisinopril | Treats high blood pressure | Blood |
| Budesonide/Formoterol | Treat asthma or ongoing lung disease - | Lung |
| Fumarate | wheezing and shortness of breath | |
| Amlodipine Besylate | Calcium channel blocker - treats high | General |
| | blood pressure and chest pain | |
| Metoprolol Tartrate | Beta blocker - treats high blood pressure | Blood |
| Omeprazole | Treat stomach and esophagus problems - | Digestion |
| | acid reflux, ulcers | |
| Insulin Glargine | Treats diabetes | Blood |
| Simvastatin | Help lower bad cholesterol | Heart |
| Escitalopram Oxalate | Treat depression and anxiety | Mental |
| Albuterol | Prevent and treat difficulty breathing, | Lung |
| | wheezing, and shortness of breath | |
| Sertraline HCl | Treat depression and panic attacks | Mental |
| Levofloxacin | Antibiotic used to treat bacterial infections | General |
| Cetirizine HCl | Antihistamine used to relieve allergy | Allergies |
| | symptoms | |
| Fluconazole | treat and prevent fungal infections | Skin |
| Dextroamphetamine/ | Treats ADHD and narcolepsy | General |
| Amphetamine | | |
| Bupropion HCl | Used to treat depression | Mental |
| Venlafaxine HCl | Used to treat depression | Mental |
| Codeine Phosphate/ | Treat cough and chest congestion from | Lung |
| Guaifenesin | allergies, cold, or flu | |
| Etonogestrel/Ethinyl | Form of contraception | Reproductive |
| Estradiol | | |
| Estrogens, Conjugated | Treat moderate or severe hot flashes, area | Reproductive |
| | of vagina, and menopause | |
| Testosterone Cypionate | Treat low levels of testosterone among | Hormone |
| | men | |
| Nicotine | Indicating Potential Nicotine Addiction | Smoke |
| | and Cessation | |
| Ipratropium/Albuterol | Opens up the airways of the lung | Lung |
| Sulfate | | |
| Pneumoc | Pneumonia Vaccine | Vaccine |
| Flu Vaccine | Flu Vaccine | Vaccine |
| Typhoid Vaccine | Typhoid Vaccine | Vaccine |
| Tetanus Toxoid | Tetanus Vaccine | Vaccine |
| Hepatitis A Virus | Hepatitis Vaccine | Vaccine |
| Vaccine | | |

In various embodiments, EHR data and claims data may include overlapping information of patients. For example, both the EHR data and claims data can include patient demographics data for patients. As another example, both the EHR data and claims data can include prior diagnoses data for patients. As another example, both the EHR data and claims data can include prior procedures data for patients. As another example, both the EHR data and claims data can include prior prescriptions data for patients. Overlapping patient data between the EHR data and claims data can be useful for verifying patient data, as the overlapping patient data would represent more reliable patient information.

In various embodiments, EHR data may include additional patient information that is not available in the claims data, and vice versa. For example, EHR data can further include additional demographics information of additional specificity that may not be available in the claims data. Such additional demographics data can include living situation (e.g., single or living alone, married, or living together) as well as language (e.g., primary spoken language). In various embodiments, EHR data can further include laboratory test data that may not be available in claims data. For example, EHR data can include measurements of characteristics or quantitative values for one or more biomarkers determined for the patient. Example laboratory test data can include values for alanine aminotransferase (ALT), body mass index, cholesterol, creatinine, forced expiratory volume (FEV-1), FEV-1/FVC ratio, glucose, high-density lipoprotein (HDL), international normalized ratio (INR), potassium, low density lipoprotein (LDL), mean corpuscular hemoglobin concentration (MCHC-M), platelets, red cell distribution width (RDW), triglycerides, white blood cells (WBC). Further examples EHR data and claims data are described in Franklin, Jessica M., et al. “The relative benefits of claims and electronic health record data for predicting medication adherence trajectory.” American heart journal 197 (2018): 153-162, which is hereby incorporated by reference in its entirety.

Altogether, in such embodiments where the EHR data and claims data differ, each dataset can be used to supplement the other dataset. Thus, using both EHR data and claims data enables the more accurate prediction and identification of candidate patients in comparison to the use of any single data alone.

### Example Feature Groupings

Methods disclosed herein further involve analyzing two or more extracted features e.g., in a feature grouping to determine whether a patient is to be categorized as a candidate patient or a non-candidate patient. By analyzing a feature grouping comprising two or more extracted features (e.g., analyzing using a trained machine learning model), methods disclosed herein can involve determining a contribution of the feature grouping that resulted in the categorization of the patient as a candidate patient or non-candidate patient.

As used herein, a “feature grouping” refers to one or more extracted features. In some embodiments, a feature grouping refers to 2 or more extracted features. A feature grouping may refer to about 2 extracted features to about 30 extracted features, without limitation. A feature grouping may refer to more than 30 extracted features, in some embodiments. For example, extracted features of a feature grouping can be related according to an anatomical organ, such as any one of a brain, heart, blood, thorax, eyes, lung, abdomen, colon, cervix, pancreas, kidney, liver, muscle, lymph nodes, oral cavity, pharynx, larynx, esophagus, intestine, spleen, stomach, and gall bladder. As another example, extracted features of a feature grouping can be related to the patient, examples of which include patient behavior, patient characteristics, smoking status, and vaccination status. Example feature groupings can include Lung, Heart, Preventative Care, Blood, Digestion, Tobacco Use/Smoking, Mental Health, Reproductive, Oral Cavity/Pharynx/Larynx, Pain/Pain Management, Health Measures/Benchmarks, and Vision.

Further exemplary feature groupings are shown below in Table 6.

TABLE 6
Example Feature Groupings of Features

| Feature | Feature Grouping | Type of Data | Description |
|---|---|---|---|
| J449 | Lung | Diagnosis Code | Chronic obstructive pulmonary disease, |
| | | | unspecified |
| J440 | Lung | Diagnosis Code | Chronic obstructive pulmonary disease with |
| | | | (acute) lower respiratory infection |
| J441 | Lung | Diagnosis Code | Chronic obstructive pulmonary disease with |
| | | | (acute) exacerbation |
| J209 | Lung | Diagnosis Code | Acute bronchitis, unspecified |
| J410 | Lung | Diagnosis Code | Simple chronic bronchitis |
| J411 | Lung | Diagnosis Code | Mucopurulent chronic bronchitis |
| J418 | Lung | Diagnosis Code | Mixed simple and mucopurulent chronic |
| | | | bronchitis |
| J42 | Lung | Diagnosis Code | Unspecified chronic bronchitis |
| J439 | Lung | Diagnosis Code | Emphysema, unspecified |
| J181 | Lung | Diagnosis Code | Lobar pneumonia, unspecified organism |
| J189 | Lung | Diagnosis Code | Pneumonia, unspecified organism |
| J4540 | Lung | Diagnosis Code | Moderate persistent asthma, uncomplicated |
| J45909 | Lung | Diagnosis Code | Unspecified asthma, uncomplicated |
| J90 | Lung | Diagnosis Code | Pleural effusion, not elsewhere classified |
| J9601 | Lung | Diagnosis Code | Acute respiratory failure with hypoxia |
| J9600 | Lung | Diagnosis Code | Acute respiratory failure, unspecified |
| | | | whether hypoxia or hypercapnia |
| J9602 | Lung | Diagnosis Code | Acute respiratory failure with hypercapnia |
| J9611 | Lung | Diagnosis Code | Chronic respiratory failure with hypoxia |
| J9621 | Lung | Diagnosis Code | Acute and chronic respiratory failure with |
| | | | hypoxia |
| J9622 | Lung | Diagnosis Code | Acute and chronic respiratory failure with |
| | | | hypercapnia |
| J984 | Lung | Diagnosis Code | Other disorders of lung |
| R05 | Lung | Diagnosis Code | Cough |
| R0600 | Lung | Diagnosis Code | Dyspnea, unspecified |
| R062 | Lung | Diagnosis Code | Wheezing |
| R600 | Lung | Diagnosis Code | Localized edema |
| R0602 | Lung | Diagnosis Code | Shortness of breath |
| Z7951 | Lung | Diagnosis Code | Long term (current) use of inhaled steroids |
| Z9981 | Lung | Diagnosis Code | Dependence on supplemental oxygen |
| R079 | Lung | Diagnosis Code | Chest pain, unspecified |
| I272 | Lung | Diagnosis Code | Other secondary pulmonary hypertension |
| I2720 | Lung | Diagnosis Code | Pulmonary hypertension, unspecified |
| Tiotropium Bromide | Lung | Prescription | Treats lung diseases - COPD or Asthma |
| Albuterol Sulfate | Lung | Prescription | Bronchodilator used to treat or prevent |
| | | | bronchospasm |
| Fluticasone Propion/ | Lung | Prescription | Treat difficulty breathing, wheezing, |
| Salmeterol | | | shortness of breath |
| Budesonide/ | Lung | Prescription | Treat asthma or ongoing lung disease - |
| Formoterol Fumarate | | | wheezing and shortness of breath |
| Albuterol | Lung | Prescription | Prevent and treat difficulty breathing, |
| | | | wheezing, and shortness of breath |
| Codeine Phosphate/ | Lung | Prescription | Treat cough and chest congestion from |
| Guaifenesin | | | allergies, cold, or flu |
| Ipratropium/ | Lung | Prescription | Opens up the airways of the lung |
| Albuterol Sulfate | | | |
| I700 | Heart | Diagnosis Code | Atherosclerosis of aorta |
| I2510 | Heart | Diagnosis Code | Atherosclerotic heart disease of native |
| | | | coronary artery without angina pectoris |
| I6523 | Heart | Diagnosis Code | Occlusion and stenosis of bilateral carotid |
| | | | arteries |
| I6521 | Heart | Diagnosis Code | Occlusion and stenosis of right carotid artery |
| I6522 | Heart | Diagnosis Code | Occlusion and stenosis of left carotid artery |
| I6529 | Heart | Diagnosis Code | Occlusion and stenosis of unspecified |
| | | | carotid artery |
| I110 | Heart | Diagnosis Code | Hypertensive heart disease with heart failure |
| I252 | Heart | Diagnosis Code | Old myocardial infarction |
| I5032 | Heart | Diagnosis Code | Chronic diastolic (congestive) heart failure |
| I5022 | Heart | Diagnosis Code | Chronic systolic (congestive) heart failure |
| I480 | Heart | Diagnosis Code | Paroxysmal atrial fibrillation |
| Z950 | Heart | Diagnosis Code | Presence of cardiac pacemaker |
| Z952 | Heart | Diagnosis Code | Presence of prosthetic heart valve |
| Z951 | Heart | Diagnosis Code | Presence of aortocoronary bypass graft |
| Z955 | Heart | Diagnosis Code | Presence of coronary angioplasty implant |
| | | | and graft |
| Simvastatin | Heart | Prescription | Help lower bad cholesterol |
| V771 | Preventative Care | Diagnosis Code | Screening for diabetes mellitus |
| 99406 | Preventative Care | Procedures Code | Behav chng smoking 3-10 min |
| 3077F | Preventative Care | Procedures Code | Syst bp >/=140 mm hg |
| 3078F | Preventative Care | Procedures Code | Diast bp <80 mm hg |
| 94060 | Preventative Care | Procedures Code | Evaluation of wheezing |
| 94640 | Preventative Care | Procedures Code | Airway inhalation treatment |
| 84520 | Preventative Care | Procedures Code | Assay of urea nitrogen |
| 99223 | Preventative Care | Procedures Code | Initial hospital care |
| J1644 | Preventative Care | Procedures Code | Inj heparin sodium per 1000 u |
| 99232 | Preventative Care | Procedures Code | Subsequent hospital care |
| 80050 | Preventative Care | Procedures Code | General health panel |
| 99396 | Preventative Care | Procedures Code | Prev visit est age 40-64 |
| 99386 | Preventative Care | Procedures Code | Prev visit new age 40-64 |
| 1036F | Preventative Care | Procedures Code | Tobacco non-user |
| 3017F | Preventative Care | Procedures Code | Colorectal ca screen doc rev |
| G8484 | Preventative Care | Procedures Code | Flu immunize no admin |
| G8752 | Preventative Care | Procedures Code | Sys bp less 140 |
| G8754 | Preventative Care | Procedures Code | Dias bp less 90 |
| Levofloxacin | Preventative Care | Prescription | Antibiotic used to treat bacterial infections |
| Cetirizine Hcl | Preventative Care | Prescription | Antihistamine used to relieve allergy |
| | | | symptoms |
| Fluconazole | Preventative Care | Prescription | treat and prevent fungal infections |
| Pneumoc | Preventative Care | Prescription | Pneumonia Vaccine |
| Flu Vacc | Preventative Care | Prescription | Flu Vaccine |
| Typhoic Vacc | Preventative Care | Prescription | Typhoid Vaccine |
| Tetanus Toxoid | Preventative Care | Prescription | Tetanus Vaccine |
| Hepatitis A Virus | Preventative Care | Prescription | Hepatitis Vaccine |
| Vaccine | | | |
| E440 | Digestion | Diagnosis Code | Moderate protein-calorie malnutrition |
| K922 | Digestion | Diagnosis Code | Gastrointestinal hemorrhage, unspecified |
| K921 | Digestion | Diagnosis Code | Melena |
| K219 | Digestion | Diagnosis Code | Gastro-esophageal reflux disease without |
| | | | esophagitis |
| Omeprazole | Digestion | Prescription | Treat stomach and esophagus problems - |
| | | | acid reflux, ulcers |
| E43 | Digestion | Diagnosis Code | Unspecified severe protein-calorie |
| | | | malnutrition |
| F17290 | Tobacco | Diagnosis Code | Nicotine dependence, other tobacco product, |
| | Use/Smoking | | uncomplicated |
| Z720 | Tobacco | Diagnosis Code | Tobacco use |
| | Use/Smoking | | |
| F17200 | Tobacco | Diagnosis Code | Nicotine dependence, unspecified, |
| | Use/Smoking | | uncomplicated |
| F17210 | Tobacco | Diagnosis Code | Nicotine dependence, cigarettes, |
| | Use/Smoking | | uncomplicated |
| Z87891 | Tobacco | Diagnosis Code | Personal history of nicotine dependence |
| | Use/Smoking | | |
| Nicotine | Tobacco | Prescription | Indicating Potential Nicotine Addiction and |
| | Use/Smoking | | Cessation |
| F489 | Mental Health | Diagnosis Code | Nonpsychotic mental disorder, unspecified |
| Escitalopram Oxalate | Mental Health | Prescription | Treat depression and anxiety |
| Sertraline Hcl | Mental Health | Prescription | Treat depression and panic attacks |
| Dextroamphetamine/ | Mental Health | Prescription | Treats ADHD and narcolepsy |
| Amphetamine | | | |
| Bupropion Hcl | Mental Health | Prescription | Used to treat depression |
| Venlafaxine Hcl | Mental Health | Prescription | Used to treat depression |
| Z01419 | Reproductive | Diagnosis Code | Encounter for gynecological examination |
| | | | (general) (routine) without abnormal |
| | | | findings |
| E291 | Reproductive | Diagnosis Code | Testicular hypofunction |
| V7231 | Reproductive | Diagnosis Code | Routine gynecological examination |
| Etonogestrel/Ethinyl | Reproductive | Prescription | Form of contraception |
| Estradiol | | | |
| Estrogens, Conjugated | Reproductive | Prescription | Treat moderate or severe hot flashes, area of |
| | | | vagina, and menopause |
| Testosterone Cypionate | Reproductive | Prescription | Treat low levels of testosterone among men |
| R490 | Oral Cavity/ | Diagnosis Code | Dysphonia |
| | Pharynx/ | | |
| | Larynx | | |
| 462 | Oral Cavity/ | Diagnosis Code | Acute pharyngitis |
| | Pharynx/ | | |
| | Larynx | | |
| G894 | Pain/ | Diagnosis Code | Chronic pain syndrome |
| | Pain Management | | |
| Z7982 | Pain/ | Diagnosis Code | Long term (current) use of aspirin |
| | Pain Management | | |
| Z7901 | Pain/ | Diagnosis Code | Long term (current) use of anticoagulants |
| | Pain Management | | |
| Z79891 | Pain/ | Diagnosis Code | Long term (current) use of opiate analgesic |
| | Pain Management | | |
| G8929 | Pain/ | Diagnosis Code | Other chronic pain |
| | Pain Management | | |
| F1120 | Pain/ | Diagnosis Code | Opioid dependence, uncomplicated |
| | Pain Management | | |
| M1990 | Pain/ | Diagnosis Code | Unspecified osteoarthritis, unspecified site |
| | Pain Management | | |
| M069 | Pain/ | Diagnosis Code | Rheumatoid arthritis, unspecified |
| | Pain Management | | |
| Z681 | Health Measures/ | Diagnosis Code | Body mass index (BMI) 19.9 or less, adult |
| | Benchmarks | | |
| Z923 | Health Measures/ | Diagnosis Code | Personal history of irradiation |
| | Benchmarks | | |
| E538 | Health Measures/ | Diagnosis Code | Deficiency of other specified B group |
| | Benchmarks | | vitamins |
| E559 | Health Measures/ | Diagnosis Code | Vitamin D deficiency, unspecified |
| | Benchmarks | | |
| Z6843 | Health Measures/ | Diagnosis Code | Body mass index (BMI) 50.0-59.9, adult |
| | Benchmarks | | |
| 7962 | Health Measures/ | Diagnosis Code | Elevated blood pressure reading without |
| | Benchmarks | | diagnosis of hypertension |
| 27801 | Health Measures/ | Diagnosis Code | Morbid obesity |
| | Benchmarks | | |
| Z961 | Vision | Diagnosis Code | Presence of intraocular lens |
| H2512 | Vision | Diagnosis Code | Age-related nuclear cataract, left eye |
| H2511 | Vision | Diagnosis Code | Age-related nuclear cataract, right eye |
| H2513 | Vision | Diagnosis Code | Age-related nuclear cataract, bilateral |

### VII. CANCERS

Methods described herein involve prioritizing medical resources by identifying candidate patients likely to be at risk of a cancer using risk prediction models. In various embodiments, the cancer in the patient can include one or more of: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, stomach cancer, thyroid cancer, head and neck carcinoma, large bowel cancer, hematopoietic cancer, testicular cancer, colon and/or rectal cancer, uterine cancer, or prostatic cancer. In some embodiments, the cancer in the patient can be a metastatic cancer, including any one of bladder cancer, breast cancer, colon cancer, kidney cancer, lung cancer, melanoma, ovarian cancer, pancreatic cancer, prostatic cancer, rectal cancer, stomach cancer, thyroid cancer, or uterine cancer. In some embodiments, the cancer is a lung cancer. In some embodiments, the cancer is a type of lung cancer, including any one of small cell lung cancer, non-small cell lung cancer, non-small cell carcinoma, adenocarcinoma, squamous cell cancer, large cell carcinoma, small cell carcinoma, combined small cell carcinoma, neuroendocrine tumor, lung sarcoma, lung lymphoma, bronchial carcinoids.

In some embodiments, the cancer is an early-stage cancer. In some embodiments, the early-stage cancer is a stage I cancer. In some embodiments, the early-stage cancer is a stage II cancer. In various embodiments, the early-stage cancer is an early-stage lung cancer. In various embodiments, the early-stage lung cancer refers to a stage prior to the development of nodules, such as lung nodules or lymph node nodules. In various embodiments, the early-stage lung cancer may not yet have been previously diagnosed or identified (e.g., via biopsy or imaging). Thus, methods disclosed herein can be useful for prioritizing patients that would most benefit from subsequent analysis (e.g., via biopsy or imaging).

### VIII. INTERVENTIONS

Embodiments described herein involve prioritizing medical resources by identifying candidate patients likely to be at risk of a cancer using risk prediction models. In various embodiments, the methods disclosed herein are performed on patients who have not previously received any of the following: an image scan (e.g., any of a LDCT/Chest-CT/PET/PET-CT scan), a lung cancer biopsy procedure, or a lung cancer diagnosis. Thus, by analyzing, in silico, electronic records (e.g., electronic health records and/or claims data) without the need for images/biopsy information, this enables rapid and cost-effective evaluation of patients to guide the provision of interventions only to candidate patients that would most likely benefit from the intervention.

In various embodiments, the intervention can be any one of: application of a diagnostic, application of a prophylactic therapeutic agent, or a subsequent action. Example subsequent actions can include a subsequent testing of the patient to confirm whether the patient develops cancer. Subsequent testing can include any of a subsequent biopsy (e.g., cancer biopsy or lymph node biopsy) or subsequent image scanning (e.g., CT scanning, PET scanning, MRI scanning, ultrasound imaging, or X-ray imaging). In some embodiments, the subsequent testing includes performing a CT or PET image scanning. The CT or PET image scanning can then be used to confirm the risk of cancer in the patient. In some embodiments, the subsequent testing includes performing a chest CT or PET image scanning.

In various embodiments, subsequent testing of the patient can occur during at a next scheduled visit or at a pre-determined amount of time such as, but not limited to, about 1 month to about 24 months after predicting the future risk of cancer. In some embodiments, a pre-determined amount of time may be less than 1 month or greater than 24 months. In various embodiments, additional subsequent actions can include subsequent actions to treat a cancer that has developed in the patient, such as tumor resection, bronchoscopic diagnosis, selection and/or administration of therapeutic(s), selection/administration of pharmaceutical composition, or any combination thereof.

In various embodiments, a therapeutic agent can be selected and/or administered to the patient based on the predicted future risk of cancer. The selected therapeutic agent is likely to delay or prevent the development of the cancer, such as lung cancer. Exemplary therapeutic agents include chemotherapies, energy therapies (e.g., external beam, microwave, radiofrequency ablation, brachytherapy, electroporation, cryoablation, photothermal ablation, laser therapy, photodynamic therapy, electrocauterization, chemoembolization, high intensity focused ultrasound, low intensity focused ultrasound), antigen-specific monoclonal antibodies, anti-inflammatoires, oncolytic viral therapies, or immunotherapies. In various embodiments, the selected therapeutic agent is an energy therapy and the amount (e.g., dose and duration) of the energy applied can be tailored to achieve a desired therapeutic effect. In various embodiments the therapeutic agent is a small molecule or biologic, e.g., a cytokine, antibody, soluble cytokine receptor, anti-sense oligonucleotide, siRNA, etc. Such biologic agents encompass muteins and derivatives of the biological agent, which derivatives can include, for example, fusion proteins, PEGylated derivatives, cholesterol conjugated derivatives, and the like as known in the art. Also included are antagonists of cytokines and cytokine receptors, e.g., traps and monoclonal antagonists. Also included are biosimilar or bioequivalent drugs to the active agents set forth herein.

Therapeutic agents for lung cancer can include chemotherapeutics such as docetaxel, cisplatin, carboplatin, gemcitabine, Nab-paclitaxel, paclitaxel, pemetrexed, gefitinib, erlotinib, brigatinib (Alunbrig®), capmatinib (Tabrecta®), selpercatinib (Retevmo®), entrectinib (Rozlytrek®), lorlatinib (Lorbrena®), larotrectinib (Vitrakvi®), dacomitinib (Vizimpro®), and vinorelbine. Therapeutic agents for lung cancer can include antibody therapies such as durvalumab (Imfinzi®), nivolumab (Opdivo®), pembrolizumab (Keytruda®), atezolizumab (Tecentriq®), canakinumab, and ramucirumab.

In various embodiments, one or more of the therapeutic agents described can be combined as a combination therapy for treating the patient.

In various embodiments, a pharmaceutical composition can be selected and/or administered to the patient based on the patient level risk of metastatic cancer, the selected therapeutic agent likely to exhibit efficacy against the cancer. A pharmaceutical composition administered to an individual includes an active agent such as the therapeutic agent described above. The active ingredient is present in a therapeutically effective amount, i.e., an amount sufficient when administered to treat a disease or medical condition mediated thereby. The compositions can also include various other agents to enhance delivery and efficacy, e.g., to enhance delivery and stability of the active ingredients. Thus, for example, the compositions can also include, depending on the formulation desired, pharmaceutically acceptable, non-toxic carriers or diluents, which are defined as vehicles commonly used to formulate pharmaceutical compositions for animal or human administration. The diluent may be selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents, and detergents. The composition can also include any of a variety of stabilizing agents, such as an antioxidant.

The pharmaceutical compositions or therapeutic agents described herein can be administered in numerous ways. Examples include administering a composition containing a pharmaceutically acceptable carrier via oral, intranasal, intramodular, intralesional, rectal, topical, intraperitoneal, intravenous, intramuscular, subcutaneous, subdermal, transdermal, intrathecal, endobronchial, transthoracic, or intracranial method.

In various embodiments, a clinical response can be provided to the patient based on the predicted future risk of cancer generated for the patient by implementing risk prediction models. In various embodiments, a clinical response can include providing counseling to modify a behavior of the patient (e.g., counsel the patient about smoking cessation to reduce risk), initiating of an inhaled/topical, intravenous or enteral (by mouth) therapeutic that could delay/prevent malignant transformation, slow tumor growth or even prevent spread of disease (metastasis), establishing an adaptive screening schedule for future risk similar to what is done with colonoscopy for polyps (e.g., individuals predicted to be higher risk for future lung cancer should have more frequent follow up and imaging), or performing or scheduling to be performed an additional risk prediction test to confirm the predicted future risk of lung cancer (e.g., persons deemed to be higher risk for lung cancer may also then undergo additional testing to either confirm that risk or narrow the cancer type the person is at greatest risk for. In various embodiments, the additional risk prediction test could include blood-based biomarkers (to look for non-specific inflammation which is a known risk for lung cancer), metabolomics/proteomics/gene expression/genetic sequencing. The person could also have additional sampling of tissue (nasal epithelium, bronchial epithelium, etc.) to look at changes in gene expression in the respiratory tract.)

### IX. COMPUTER IMPLEMENTATION

The methods disclosed herein, including the prioritizing medical resources by identifying candidate patients likely to be at risk of a cancer using risk prediction models, are, in some embodiments, performed on one or more computers. For example, as shown in reference to FIGS. 1A and 1B, the patient prioritization system 130 can include one or more computers. Therefore, in various embodiments, the steps described in reference to the patient prioritization system 130 are performed in silico.

In various embodiments, the building and deployment of a risk prediction model can be implemented in hardware or software, or a combination of both. In one embodiment of the disclosure, a machine-readable storage medium is provided, the medium comprising a data storage material encoded with machine readable data which, when using a machine programmed with instructions for using said data, is capable of executing the training or deployment of risk prediction models and/or displaying any of the datasets or results (e.g., future risk of cancer predictions for patients) described herein. The disclosure can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present disclosure. The databases of the present disclosure can be recorded on computer readable media, e.g., any medium that can be read and accessed directly by a computer. Such media include, but are not limited to magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g., word processing text file, database format, etc.

In some embodiments, the methods of the disclosure, including the methods of prioritizing medical resources by identifying candidate patients, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

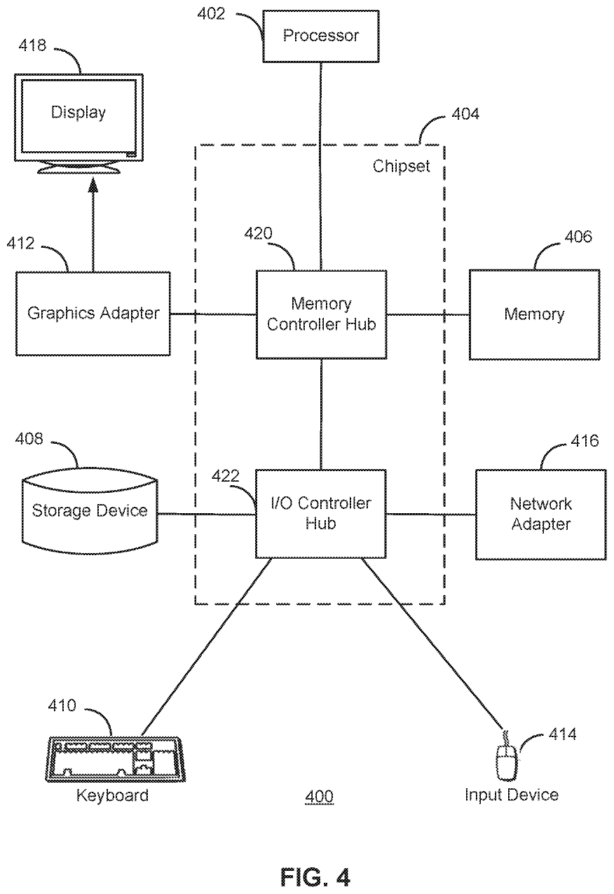

FIG. 4 illustrates an example computer for implementing the entities shown in FIGS. 1A, 1B, 2, 3A, and 3B. The computer 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input device 414, and network adapter 416 are coupled to the I/O controller hub 422. Other embodiments of the computer 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input device 414 is a touch-screen interface, a mouse, track ball, or other type of pointing device, a keyboard, or some combination thereof, and is used to input data into the computer 400. In some embodiments, the computer 400 may be configured to receive input (e.g., commands) from the input device 414 via gestures from the user. The network adapter 416 couples the computer 400 to one or more computer networks.

The graphics adapter 412 displays images and other information on the display 418. In various embodiments, the display 418 is configured such that the user may (e.g., radiologist, oncologist, pulmonologist) may input user selections on the display 418 to, for example, initiate risk prediction for a patient, order any additional exams or procedures and/or set parameters for the risk prediction models. In one embodiment, the display 418 may include a touch interface.

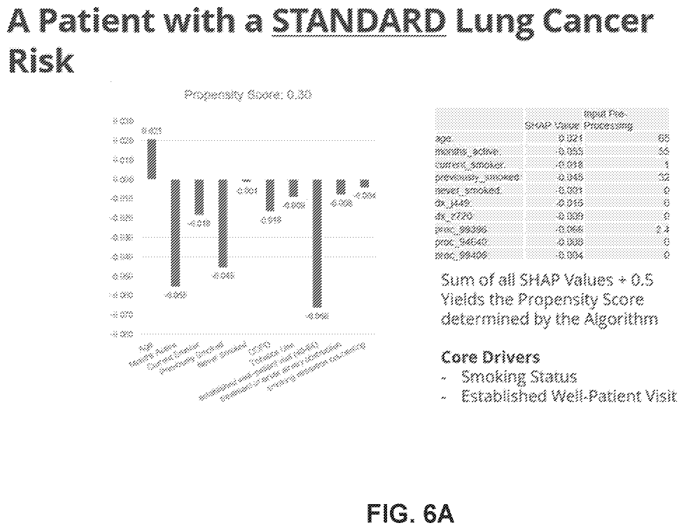

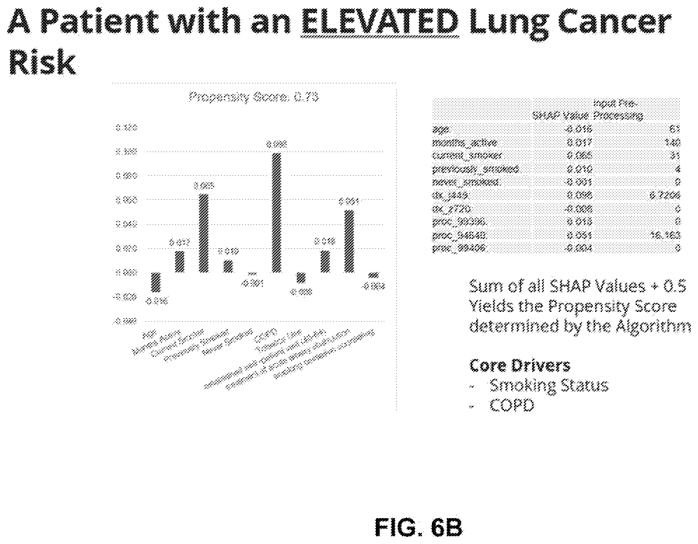

In various embodiments, the display 418 can show one or more predictions of a risk prediction model. For example, the display 418 can show a score indicative of lung cancer risk for the patient. As another example, the display 418 can show scores for feature groupings that contribute to the score indicative of lung cancer risk. Example information shown on a display 418 are depicted in FIGS. 6A and 6B, and described in further detail below.

A user who accesses the display 418 can inform the patient of the score indicative of lung cancer risk. In various embodiments, the display 418 can show information such as the feature groupings that most heavily contributed to the score indicative of lung cancer risk. Displaying the top contributing feature groups can provide context to a user e.g., clinician user in understanding the features that resulted in the score indicative of lung cancer.

The computer 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computers 400 can vary depending upon the embodiment and the processing power required by the entity. For example, the patient prioritization system 130 can run in a single computer 400 or multiple computers 400 communicating with each other through a network such as in a server farm. The computers 400 can lack some of the components described above, such as graphics adapters 412 and displays 418.

Further disclosed herein are systems for prioritizing medical resources by identifying candidate patients. In various embodiments, such a system can include at least the patient prioritization system 130 described above in FIG. 1A. In various embodiments, the patient prioritization system 130 is embodied as a computer system, such as a computer system with example computer 400 described in FIG. 4.

### EXAMPLES

Below are example embodiments for carrying out the present disclosure. The examples are offered for illustrative purposes only and are not intended to limit the scope of the present disclosure in any way.

### Example 1: Example Categorization of Patients

Example 1 describes an algorithm to identify candidate patients. It is noted that the current USPSTF recommendations are based on certain factors. For example, the USPSTF recommends that patients who meet data points of between 50-80 years old and with a 20+ smoking pack year history to pursue a preliminary low dose computed tomography (LDCT) scan for possible lung cancer. However, there are drawbacks using the USPSTF recommendations—for example, the 20+ smoking pack year history is largely a self-reported data point and its accuracy is dependent on the patients reporting the correct number if they report at all.

Using the data from United States Preventive Services Task Force (USPSTF) Recommendations, there were an estimated 90 million current and former smokers across all ages. 45 million current and former smokers are between the ages of 50-80 years old, with only 15 million of those individuals having a 20 pack-year smoking history. The related statistics using the USPSTF Recommendations is as follows: One-year positive predictive value (PPV) of 0.62% and a population incidence of 0.21%.

Comparatively, the machine learning algorithm in accordance with this disclosure analyzed patients between 50-80 years old with a smoking related observation who also have not received any of the following:
a LDCT/Chest-CT/PET/PET-CT scan,
a lung cancer biopsy procedure, or
a lung cancer diagnosis.

At a high level, the machine learning algorithm identifies candidate patients at risk of lung cancer. Various machine learning models can be used (the results of which are described below in Example 2). For example, machine learning models may be developed using a gradient boosting decision tree algorithm such as CatBoost or XGBoost. Other machine learning models may be developed, e.g., a neural network. The algorithm may be trained on features extracted from stored electronic data (e.g., lung issues, heart issues) and claims data (e.g., procedure codes), and may be analyzed through Shapley analysis. Each third party, e.g., clinical site, can adjust a threshold for demarcating elevated versus standard risk of lung cancer, based on site preference. Once the threshold has been set, the patient's electronic health records and/or claims data may be used as input. The raw output of the algorithm may be a floating integer (propensity score) between 0 and 1 corresponding to lung cancer risk score. Higher number may refer to higher risk. The raw output may also include a list of “features” (each “feature” is either an individual feature, or a feature grouping of related features to ease Shapley computational burden) and their Shapley additive contribution to lung cancer score. The formatted output may be visible to the user and may be a binary output of elevated versus standard risk. The user interface may also show feature(s) that contributed most to the score.

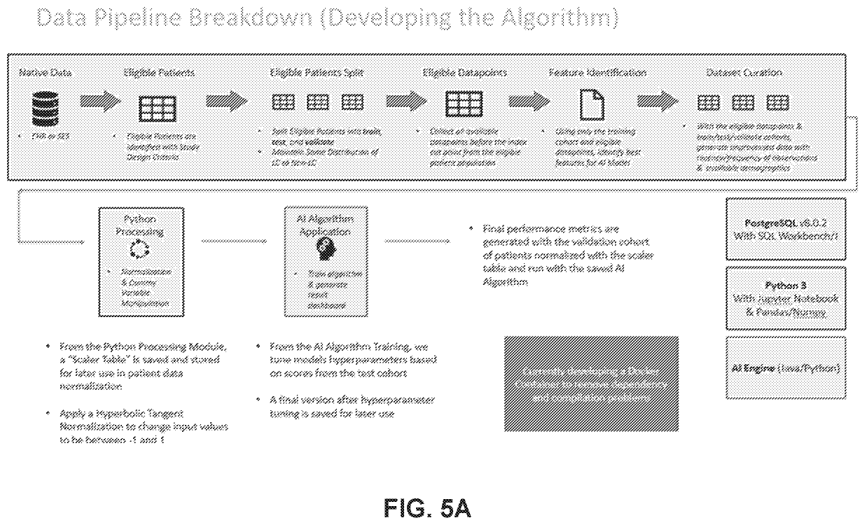

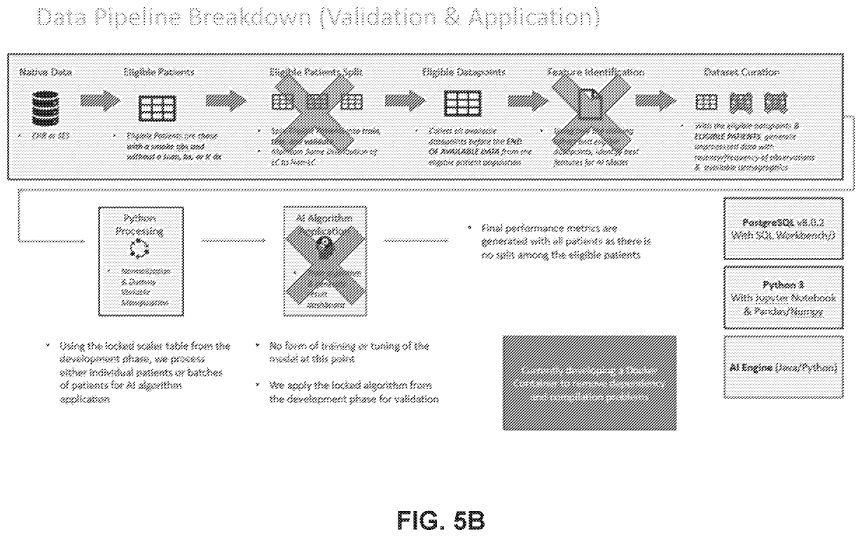

FIG. 5A depicts an example data pipeline for developing the algorithm (e.g., machine learning model). Native electronic data (e.g., electronic health record (EHR) or claims data) were retrieved and eligible patient with the study criteria (e.g., 50-80 years old, smoking related observation, and no prior scan, biopsy procedure, or cancer diagnosis) were identified. Here, the selected patient data can be provided (from health care provider or parsed from health care provider output) in the form of four tables listing the following info for each patient:
diagnosis (Dx) codes (e.g., lung issues such as COPD, chronic bronchitis, pneumonia, emphysema; heart issues such as hypertension, vascular disease, hyperlipidemia)
procedure codes
prescription (NDC) codes
patient demographic codes

Patients may be split into training, testing, and validation datasets. Using the training cohort, the data underwent feature identification. Furthermore, the patient data may undergo a transformation (e.g., a hyperbolic transformation) to change input values to be between −1 and 1. Next, the patient data may be used to train the machine learning models. The parameters and/or hyperparameters of the machine learning models may be tuned during training and final versions of the models may be saved after training.

Following training, the machine learning models may undergo further validation. Reference is now made to FIG. 5B, which depicts an example data pipeline for validating the algorithm (e.g., machine learning model). In this case, no further training or tuning of the machine learning models occurred in this phase. Using patient data from patients categorized in the validation set or testing set, the machine learning models may be deployed to determine final performance metrics, measuring the performance of the machine learning models.

Specifically, software may process patient data into “scalar tables.” The patient data may be input into algorithm and the algorithm may analyze designated input features (diagnoses, procedures, prescriptions, demographics). The example features are described herein in Tables 1-5.

The raw output of the algorithm may include:
“Propensity score” or “lung cancer score” (e.g., normalized to 0-1, or any other suitable scale). The lung cancer score may be compared to a predetermined threshold to classify a patient as having an elevated risk or a standard risk for future lung cancer.
Shapley additive contribution to lung cancer score for each feature or feature grouping.

The output to a health care provider may include:
Classification of patient as having elevated or standard risk
Top 3 features or feature groupings contributing most to the lung cancer score per their Shapley contributions

For example, FIGS. 6A and 6B depict example outputs for a patient with a standard lung cancer risk and a patient with an elevated lung cancer risk, respectively. FIG. 6A shows the results of a patient with standard lung cancer risk (e.g., a non-candidate patient). Here, the machine learning model predicted an overall score for the patient (e.g., “propensity score”) of 0.30. Given that the score is below a threshold value, the patient is categorized as a non-candidate patient. The chart on the left as well as the table on the right in FIG. 6A shows individual contributions of various features. The contributions are denoted as “SHAP values.” The “core drivers” shown in FIG. 6A indicates the most influential and important feature groupings that contributed to the propensity score.

FIG. 6B shows the results of a patient with elevated lung cancer risk (e.g., a candidate patient). Here, the machine learning model predicted an overall score for the patient (e.g., “propensity score”) of 0.73. Given that the score is above a threshold value, the patient is categorized as a candidate patient. The chart on the left as well as the table on the right in FIG. 6B show individual contributions of various features. The contributions are denoted as “SHAP values.” The “core drivers” shown in FIG. 6B of Smoking Status and COPD indicate the most influential and important feature groupings that contributed to the propensity score.

Altogether, the candidate patient corresponding to the results in FIG. 6B can be prioritized for subsequent screening (e.g., imaging, such as a subsequent CT scan) whereas the non-candidate patient corresponding to the results in FIG. 6A can be withheld from subsequent screening.

### Example 2: Categorization of Patients Using Logistic Regression, Neural Network, XGBoost, and CatBoost Machine Learning Models

Various machine learning models may be developed to show the applicability of the disclosed methodology across different machine learning model architectures. Using the methodology described in Example 1, each of a logistic regression machine learning model, a neural network machine learning model, a XGBoost gradient boosted decision tree, and CatBoost gradient boosted decision tree may be developed.

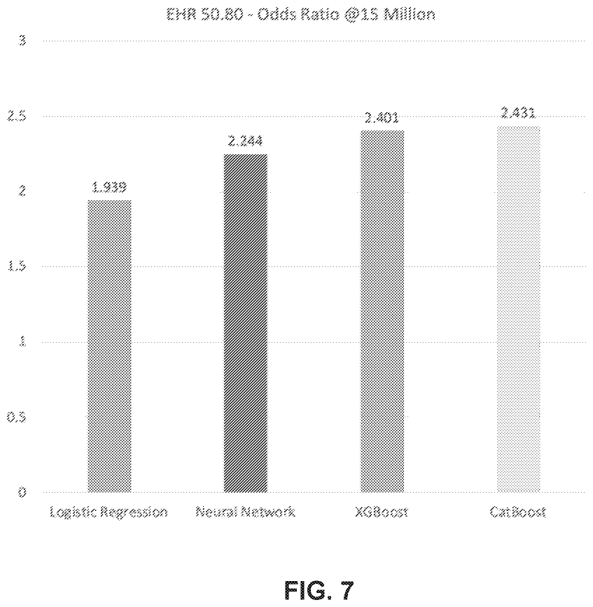

Reference is now made to FIG. 7, which shows performance of various machine learning models (e.g., logistic regression, neural network, XGBoost, and CatBoost). As shown in FIG. 7, each of the machine learning models identified patients that were of high risk of developing lung cancer. The various machine learning approaches may provide varying results. For example, FIG. 7 shows that gradient boosting decision tree algorithms (CatBoost, XGBoost) appears to provide the best results, followed by neural network, and logistic regression. However, each machine learning model achieved an Odds Ratio (e.g., Odds Ratio refers to the relative risk of the high-risk population compared to the standard risk) greater than 1, indicating that all machine learning models were successful.

1-121. (canceled)

122. A method for prioritizing patients, the method comprising:
obtaining by a computing device, a dataset comprising electronic health records of a patient stored on a database in communication with the computing device;
extracting, by the computing device, features from the electronic health records, wherein the features include at least one of demographic data, clinical data, or imaging data;
storing, by the computing device, a predefined threshold value indicative of elevated lung cancer risk;
analyzing the features by the computing device using a trained machine learning model to classify the patient as a candidate patient with a higher risk of lung cancer or a non-candidate patient with a lower risk of lung cancer using a score based on the features and the predefined threshold value; and
displaying by the computing device, an indication of the patient's predicted lung cancer risk on a display in communication with the computing device.

123. The method of claim 122 further comprising:
displaying by the computing device, a score indicative of lung cancer risk for the patient on the display in communication with the computing device and the predefined threshold value wherein the score indicative of lung cancer risk for the patient is greater than the predefined threshold value.

124. The method of claim 122 further comprising:
displaying by the computing device, a feature grouping that contributed to the score indicative of lung cancer risk, wherein the feature grouping comprises two or more features on the display in communication with the computing device.

125. The method of claim 122 further comprising:
providing by the computing device, identification of the candidate patient at risk for lung cancer and the identification of the feature grouping to a third party computing device for prioritization of medical resources.

126. The method of claim 122, wherein the extracted features comprises one or more of:
tobacco use, nicotine dependence, cigarette use, smoking cessation, number of months actively smoking, never smoked observation, and current smoker observation.

127. The method of claim 122, wherein the extracted features comprises one or more of chronic obstructive pulmonary disease (COPD), chronic bronchitis, pleural effusion, dyspnea, wheezing, and inhaled treatment for COPD and/or asthma.

128. The method of claim 122, further comprising:
selecting the predefined threshold value by the computing device, according to a received indication of available medical resources of a third party.

129. The method of claim 122, further comprising:
selecting by the computing device, a therapeutic agent for the candidate patient with the higher risk of lung cancer for delaying or preventing the development of lung cancer, wherein the therapeutic agent comprises one or more of chemotherapies, energy therapies, antigen-specific monoclonal antibodies, anti-inflammatories, oncolytic viral therapies, and immunotherapies.

130. The method of claim 122, further comprising:
selecting by the computing device, a pharmaceutical composition for the candidate patient with the higher risk of lung cancer for delaying or preventing the development of lung cancer, wherein the therapeutic agent comprises one or more of chemotherapies, energy therapies, antigen-specific monoclonal antibodies, anti-inflammatories, oncolytic viral therapies, and immunotherapies.

131. The method of claim 122, further comprising:
weighting the features by the computing device, according to timepoints that the features were recorded in the electronic records of the patient, wherein higher weights are assigned to features that were more recently recorded in the electronic health records than the features that were earlier recorded in the electronic health records.

132. A method for prioritizing patients, the method comprising:
obtaining, by the computing device, features related to lung health from the electronic health records of a patient stored on a database in communication with the computing device, wherein the features are;
analyzing the features by the computing device using a trained machine learning model to classify the patient as a candidate patient with a higher risk of lung cancer or a non-candidate patient with a lower risk of lung cancer using a score based on the extracted features and a predefined threshold value indicative of elevated lung cancer risk; and
displaying by the computing device, a score indicative of lung cancer risk for the patient on the display in communication with the computing device and the predefined threshold value wherein the score indicative of lung cancer risk for the patient is greater than the predefined threshold value.

133. The method of claim 132 further comprising:
displaying by the computing device, a feature grouping that contributed to the score indicative of lung cancer risk, wherein the feature grouping comprises two or more features on the display in communication with the computing device.

134. The method of claim 132 further comprising:
providing by the computing device, identification of the candidate patient at risk for lung cancer and the identification of the feature grouping to a third party computing device for prioritization of medical resources.

135. The method of claim 132, wherein the extracted features comprises one or more of: tobacco use, nicotine dependence, cigarette use, smoking cessation, number of months actively smoking, never smoked observation, and current smoker observation.

136. The method of claim 132, wherein the extracted features comprises one or more of chronic obstructive pulmonary disease (COPD), chronic bronchitis, pleural effusion, dyspnea, wheezing, and inhaled treatment for COPD and/or asthma.

137. The method of claim 132, further comprising:
selecting the predefined threshold value by the computing device, according to a received indication of available medical resources of a third party.

138. The method of claim 132, further comprising:
selecting by the computing device, a therapeutic agent for the candidate patient with the higher risk of lung cancer for delaying or preventing the development of lung cancer, wherein the therapeutic agent comprises one or more of chemotherapies, energy therapies, antigen-specific monoclonal antibodies, anti-inflammatories, oncolytic viral therapies, and immunotherapies.

139. The method of claim 132, further comprising:
selecting by the computing device, a pharmaceutical composition for the candidate patient with the higher risk of lung cancer for delaying or preventing the development of lung cancer, wherein the therapeutic agent comprises one or more of chemotherapies, energy therapies, antigen-specific monoclonal antibodies, anti-inflammatories, oncolytic viral therapies, and immunotherapies.

140. The method of claim 132, further comprising:
weighting the features by the computing device, according to timepoints that the features were recorded in the electronic records of the patient, wherein higher weights are assigned to features that were more recently recorded in the electronic health records than the features that were earlier recorded in the electronic health records.
